(19) United States
(12) Patent Application Publication
(10) Pub. No.: US 2020/0015686 A1
(43) Pub. Date: Jan. 16, 2020

(54) PULSE WAVE MEASURING DEVICE, SYSTEM, AND METHOD

(71) Applicants: Kazuki Funahashi, Kanagawa (JP); Atsushi Takaura, Tokyo (JP); Yasuo Miyoshi, Kanagawa (JP)

(72) Inventors: Kazuki Funahashi, Kanagawa (JP); Atsushi Takaura, Tokyo (JP); Yasuo Miyoshi, Kanagawa (JP)

(73) Assignee: Ricoh Company, Ltd., Tokyo (JP)

(21) Appl. No.: 16/578,744

(22) Filed: Sep. 23, 2019

Related U.S. Application Data
(62) Division of application No. 15/357,618, filed on Nov. 21, 2016.

(30) Foreign Application Priority Data
Nov. 27, 2015 (JP) 2015231946
Nov. 30, 2015 (JP) 2015233683
Jun. 30, 2016 (JP) 2016131001

(51) Int. Cl. A61B 5/024; A61B 5/0285

(52) U.S. Cl. CPC A61B 5/024; A61B 5/0285; A61B 5/02438

(57) ABSTRACT

A pulse wave measuring device includes a signal divider configured to divide a first corrected signal and a second corrected signal into a plurality of segments, respectively, each of which corresponds to a cycle of a pulse wave of a living body, the first corrected signal and the second corrected signal being results obtained through a first correction and a second correction respectively using a first signal and a second signal that are extracted from image data and that change in association with the pulse wave; an influence degree calculator configured to calculate a value indicating a degree of a noise influence in each of the first corrected signal and the second corrected signal for each of the plurality of segments; and a peak time detector configured to detect a time of occurrence of a peak in one of the first corrected signal or the second corrected signal.

### CROSS-REFERENCE TO RELATED APPLICATIONS

The present application claims the benefit of priority under 35 U.S.C. § 119 of Japanese Patent Application No. 2015-231946 filed on Nov. 27, 2015 in the Japan Patent Office, Japanese Patent Application No. 2015-233683 filed on Nov. 30, 2015 in the Japan Patent Office, and Japanese Patent Application No. 2016-131001 filed on Jun. 30, 2016 in the Japan Patent Office, the entire contents of which are incorporated herein by reference. The present application is a divisional of co-pending U.S. patent application Ser. No. 15/357,618, filed on Nov. 21, 2016 and identically titled, which is hereby incorporated by reference.

### BACKGROUND OF THE INVENTION

### 1. Field of the Invention

The disclosures herein generally relate to a pulse wave measuring device, a pulse wave measuring system, and a pulse wave measuring method for measuring pulse waves.

### 2. Description of the Related Art

A technique of measuring the pulse of a living body using an optical sensor or an imaging device is known as one technique. In this technique, various methods are used for improving the accuracy of detecting the pulse.

In one of the methods, in measuring the pulse using an imaging device, for example, three signals acquired from the imaging device are converted into two differential signals so as to reduce in-phase noise components. In another one of the methods, the frame rate of the imaging device is controlled so as to minimize the error between the pulse rate calculated from the signal acquired from the imaging device and the true pulse rate.

It is understood that the operation of the autonomic nerve in a living body is associated with a change of the period between peaks of pulse waves. In order to analyze the operation of the autonomic nerve, there is a need for accurately measuring the change of the period between the peaks of pulse waves.

The above-described related art, however, has the purpose of improving the accuracy of detecting the pulse rate that is the number of the peaks of pulse waves. For the purpose of measuring a change of the period between the peaks of pulse waves, the accuracy of detecting the peak is insufficient.

### SUMMARY OF THE INVENTION

It is a general object of at least one embodiment of the present invention to provide a pulse wave measuring device, a recording medium for storing a pulse wave measuring program, a pulse wave measuring method, and a pulse wave measuring system for measuring pulse waves that substantially obviate one or more problems caused by the limitations and disadvantages of the related art.

In one embodiment, a pulse wave measuring device includes a signal divider configured to divide a first corrected signal and a second corrected signal into a plurality of segments, respectively, each of which corresponds to a cycle of a pulse wave of a living body, the first corrected signal and the second corrected signal being results obtained through a first correction and a second correction respectively using a first signal and a second signal that are extracted from image data and that change in association with the pulse wave; an influence degree calculator configured to calculate a value indicating a degree of a noise influence in each of the first corrected signal and the second corrected signal for each of the plurality of segments; and a peak time detector configured to detect, separately for each of the plurality of segments, a time of occurrence of a peak in one of the first corrected signal and the second corrected signal that has a smaller degree of the noise influence for each of the plurality of segments.

### BRIEF DESCRIPTION OF THE DRAWINGS

FIG. 1 illustrates measurement of the pulse wave;

FIG. 2 is a diagram illustrating an example of the hardware configuration of a pule wave measuring device;

FIG. 3 is a diagram illustrating a functional configuration of the pulse wave measuring device in a first embodiment;

FIG. 4 is an example of the RGB signals;

FIG. 5 illustrates an example of a differential correction result;

FIG. 6A and FIG. 6B illustrate an example of a filter correction result;

FIG. 7 is a flowchart of an operation of the pulse wave measuring device;

FIG. 8 illustrates acquisition of an image by the imaging unit;

FIG. 9 is a flowchart of a process of a signal extractor;

FIG. 10 is a flowchart of a process of a peak detection processing unit in the first embodiment;

FIG. 11 is a diagram illustrating a process of calculating an influence degree;

FIG. 12 is a diagram illustrating advantages of the first embodiment;

FIG. 13 is a flowchart of a process of a peak detection processing unit in a second embodiment;

FIG. 14 is a flowchart of a process of a peak detection processing unit in a third embodiment;

FIG. 15 is a diagram illustrating a functional configuration of the pulse wave measuring device in a fourth embodiment;

FIG. 16 illustrates a pulse wave measuring system in a fifth embodiment;

FIG. 17 is a block diagram illustrating an example of a functional configuration of the pulse wave measuring device in a sixth embodiment;

overall process of the pulse wave measuring device in the sixth embodiment;

FIG. 19 is a diagram illustrating an example of the pulse wave signal in the sixth embodiment;

FIG. 20 is a flowchart of an example of the process of calculating the SN ratio in the sixth embodiment;

FIG. 21 is a graph of an example of a power spectrum calculated in calculation of the SN ratio in the sixth embodiment;

FIG. 22 is a graph of a setting example of a threshold value in the sixth embodiment;

FIG. 23A and FIG. 23B are timing diagrams illustrating an example of control by the pulse wave measuring device in the sixth embodiment;

FIG. 24 is a flowchart of an example of analysis and correction by the wave pulse measuring device in the sixth embodiment;

FIG. 25 illustrates a correction example of the peak time by the pulse wave measuring device in the sixth embodiment; and

FIG. 26 illustrates another correction example of the peak time by the pulse wave measuring device in the sixth embodiment.

### DETAILED DESCRIPTION OF THE PREFERRED EMBODIMENTS

In the following, embodiments of the present invention will be described with reference to the accompanying drawings.

### First Embodiment

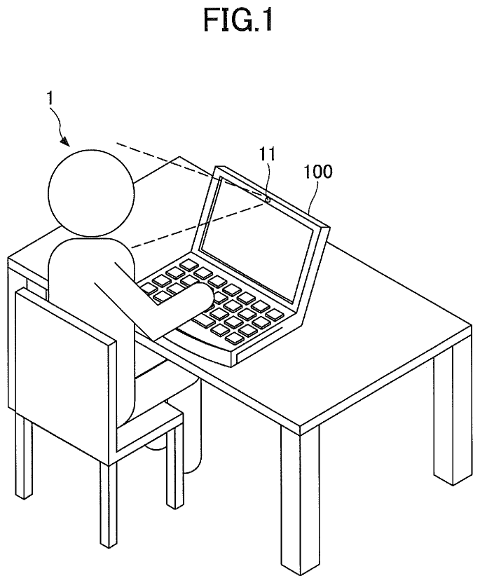

Hereinafter, a first embodiment will be described with reference to the drawings. FIG. 1 illustrates measurement of the pulse wave.

In the present embodiment, an imaging device (e.g., camera) 11 embedded in a pulse wave measuring device 100 captures images of a living body 1, which is a measurement target from which the pulse waves are taken. The imaging device 11 acquires signals indicating the pulse waves of the living body 1 from the image data of the captured image. The pulse wave measuring device 100 in the present embodiment detects the time of occurrence of each peak in the signal (e.g., biological signal) indicating the pulse wave, in a peak detection process to be described later.

More specifically, in the present embodiment, a plurality of correction processes are performed on the signals indicating the pulse waves. From a plurality of corrected signals that have been obtained as a result of the plurality of correction processes, a corrected signal with the smallest noise influence is determined. By using such a corrected signal with the smallest noise influence, the time of occurrence of the peak in the signal indicating the pulse wave is detected. Thus, in the present embodiment, the accuracy of detecting the peak of the pulse wave is improved.

Additionally, in the present embodiment, the improved accuracy of detecting the peak also improves the accuracy of detecting the change of the period between the peaks. Therefore, the present embodiment allows highly accurate information indicating the change of a period between pulses to be available to a device that performs a process using such information. This configuration contributes to the improvement in the reliability of process results. Examples of the process using the information indicating the change of the period between the peaks may include, but are not limited to, a process of analyzing the operation of the autonomic nerve in a living body.

The pulse wave measuring device 100 in the present embodiment may be any device that includes an imaging device. To be specific, as illustrated in FIG. 1, the pulse wave measuring device 100 may be a computer in which the imaging device such as a Web camera is embedded. The pulse wave measuring device 100 in the present embodiment may not include an embedded imaging device. The pulse wave measuring device 100 may include a generally-used Web camera and a computer coupled to the Web camera.

The pulse wave measuring device 100 in the present embodiment may be a mobile terminal such as a tablet computer or a smartphone, for example. The pulse wave measuring device 100 in the present embodiment may be a wearable terminal, for example, in which an imaging device is embedded or to which an imaging device is coupled.

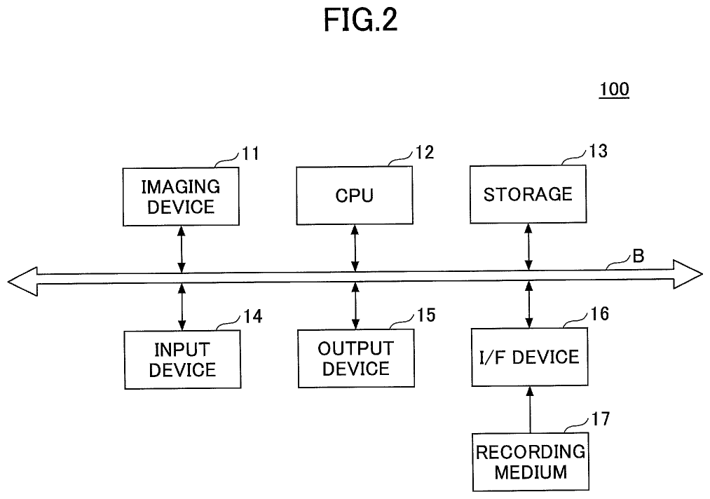

Hereinafter, a hardware configuration of the pulse wave measuring device 100 in the present embodiment will be described. FIG. 2 is a diagram illustrating an example of the hardware configuration of the pulse wave measuring device.

The pulse wave measuring device 100 in the present embodiment includes an imaging device 11, a Central Processing Unit (CPU) 12, a storage 13, an input device 14, an output device 15, and an interface (I/F) device 16. In the pulse wave measuring device 100, the imaging device 11, the CPU 12, the storage 13, the input device 14, the output device 15, and the I/F device 16 are coupled to one another via a system bus B.

The imaging device 11 includes a Charge-Coupled Device (CCD) image sensor with three channels of Red (R), Green (G), and Blue (B). The imaging device 11 acquires an RGB image of the living body 1. In the imaging device 11, the three channels of RGB may not be necessarily provided. In the imaging device 11, at least two channels having spectral sensitivities different from each other may be provided. In such a case, the two channels may include a signal value (hereinafter, referred to as signal G) of a channel having a spectral sensitivity in the G-wavelength region where the absorption rate of hemoglobin is high (i.e., the wavelength region around 550 nanometers), and a signal value of a channel having a spectral sensitivity in a wavelength region where the absorption rate of hemoglobin is low, other than the G-wavelength region. The imaging element of the imaging device 11 may be a Complementary MOS (CMOS) image sensor.

The CPU 12 is a central processing unit that controls the entirety of the pulse wave measuring device 100 and that performs operations of the pulse wave measuring device 100. The CPU 12 reads out a program stored in the storage 13, and runs the program so as to detect the peak in each pulse of the pulse wave and the period between the peaks from the image that has been acquired by the imaging device 11.

The storage 13 includes a Random Access Memory (RAM), a Read Only Memory (ROM), and an external memory device. The storage 13 stores data needed for programs and operations performed by the CPU 12.

The input device 14 is a keyboard or a mouse, for example, and is used for inputting various types of information. For example, the input device 14 is used for inputting an instruction of performing a pulse wave measurement process.

The output device 15 is a display, for example, and is used for outputting various types of information. For example, the output device 15 is used for outputting the image that has been captured by the imaging device 11 or the pulse wave data that have been measured.

The I/F device 16 includes a general-purpose interface (I/F) to be coupled to an external device. The I/F device 16 may be coupled to a recording medium 17 in which a pulse wave measuring program in the present embodiment is stored, for example. The pulse wave measuring program in the present embodiment is part of the program that controls the pulse wave measuring device 100. The pulse wave measuring program stored in the recording medium 17 is read out via the I/F device 16, and is then stored in the storage 13. The pulse wave measuring program in the present embodiment may be downloaded from another device coupled through a network, for example.

In the example of FIG. 2, the pulse wave measuring device 100 includes the imaging device 11, but is not limited to this configuration. The imaging device 11 may be externally coupled through the I/F device 16, for example.

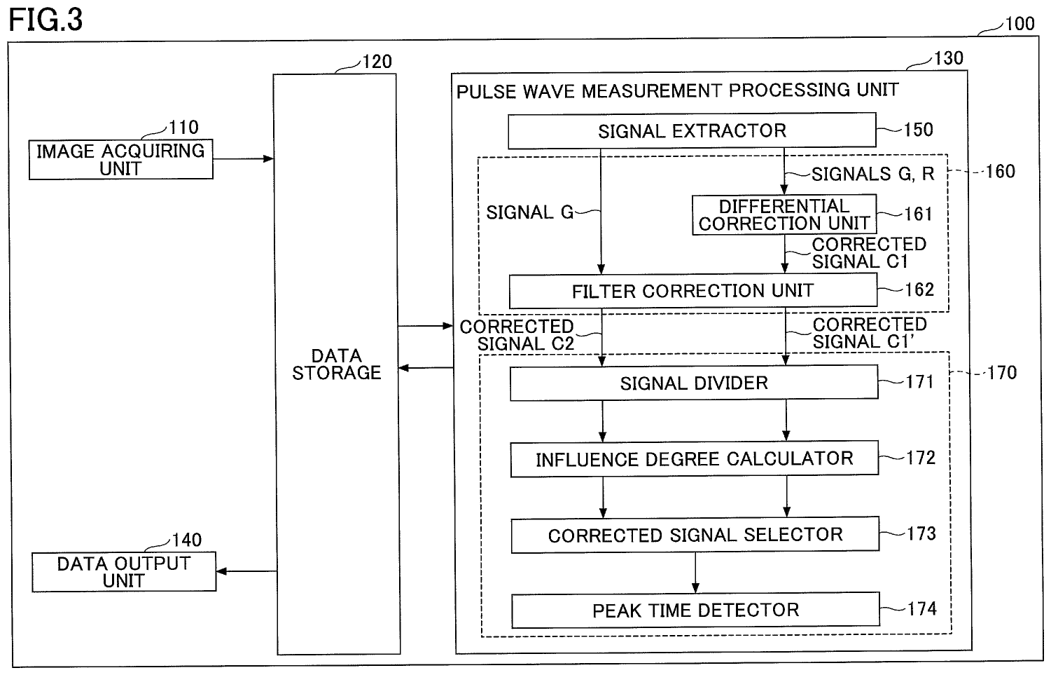

FIG. 3 is a diagram illustrating a functional configuration of the pulse wave measuring device in the first embodiment. The pulse wave measuring device 100 in the present embodiment includes an image acquiring unit 110, a data storage 120, a pulse wave measurement processing unit 130, and a data output unit 140.

The image acquiring unit 110 acquires image data of an RGB image that has been captured by the imaging device 11. To be specific, the image acquiring unit 110 in the present embodiment causes the imaging device 11 to acquire the image data with the frame rate set at 30 fps and the number of bits of RGB signal set at 8 bits. In the present embodiment, optimal values of parameters such as the frame rate and the number of bits depend on the measurement environment, the performance of the imaging device 11, and subsequent data processing. Hence, optimal values of these parameters may be set in consideration of the influences of these factors.

The data storage 120 stores in a memory area in the storage 13 the image data that have been acquired by the image acquiring unit 110 and the pulse wave data that have been output from the pulse wave measurement processing unit 130. The data storage 120 in the present embodiment stores an image data group that has been acquired by the image acquiring unit 110 at a predetermined measurement time, for example.

The pulse wave measurement processing unit 130 acquires the image data group stored in the data storage 120, and outputs the pulse wave data. The pulse wave measurement processing unit 130 will be described later in detail.

The data output unit 140 outputs the pulse wave data stored in the data storage 120 to the output device device 15 or to an external device.

Hereinafter, the pulse wave measurement processing unit 130 in the present embodiment will be described. The pulse wave measurement processing unit 130 in the present embodiment includes a signal extractor 150, a correction unit 160, and a peak detection processing unit 170.

The signal extractor 150 acquires a signal indicating a pulse wave from the data of each image in the image data group stored in the data storage 120.

In the following, how to acquire the signal indicating the pulse wave will be described. Hemoglobin contained in the blood of the living body 1 has a property of absorbing the light of a certain wavelength. The light absorption amount changes depending on a change in the hemoglobin amount in the blood induced by a change in vessel volume of blood. Such a cyclic change is found in a change in the light amount reflected from the living body 1 that is received by a receiving element of the imaging device 11. In other words, such a cyclic change is found in the change in the color of the image to be captured by the imaging device 11. Since a change in the vessel volume of blood is made by the pulse of the living body 1, the signals (i.e., signal R, signal G, and signal B) that have been extracted from the image data serve as the signals indicating the pulse waves.

The signal extractor 150 in the present embodiment extracts from the image data a signal value (hereinafter, referred to as signal G) of a channel having a spectral sensitivity in the G-wavelength region where the hemoglobin absorption rate is high (i.e., the wavelength region around 550 nanometers) and a signal value (hereinafter, referred to as signal R) of a channel having a spectral sensitivity in an R-wavelength region. Then, the signal extractor 150 in the present embodiment outputs the extracted signal values to the correction unit 160. The signals extracted by the signal extractor 150 and the process of the signal extractor 150 will be described later in detail.

It is to be noted that in the above description, the signal extractor 150 is configured to extract the signal R and the signal G, but is not limited to this configuration. The signal extractor 150 may extract a signal value (hereinafter, referred to as signal B) of a channel having a spectral sensitivity in a B-wavelength region.

The correction unit 160 performs several types of correction on the signal G and the signal R that have been extracted by the signal extractor 150 from the data of each image in the image data group. The correction unit 160 in the present embodiment includes a differential correction unit 161 and a filter correction unit 162.

The differential correction unit 161 in the present embodiment calculates the difference between the signal R and the signal G, and generates a corrected signal in which the in-phase noise between the two signals has been reduced.

The differential correction unit 161 in the present embodiment performs the differential correction in the following expression (1) using the signal G and the signal R to calculate a corrected signal C1 in which the differential correction has been performed. Examples of the corrected signal C1 will be described later. It is to be noted that “k” in the expression (1) is a coefficient for correcting a sensitivity difference between the signal G and the signal B.

C1=G-kR (1)

In the present embodiment, the corrected signal C1 is calculated by using the signal G and the signal R, but is not limited to the signal G and the signal R. The two signals used for acquiring the difference may be any other combination that is selected from the RGB signals, when the corrected signal C1 in which the in-phase noise component has been reduced to be smaller than the signal G is obtainable. In this case, one of the signals used for acquiring the difference may be the signal G, which is the signal in the G-wavelength region where the hemoglobin absorption rate is high.

In the present embodiment, the differential correction unit 161 corrects the signal that has been extracted by the signal extractor 150, so that the in-phase noise component having the same cycle with the cycle of the pulse wave can be reduced. Therefore, the differential correction unit 161 is capable of suppressing the occurrence of failure to detect, or erroneously detecting a pulse wave peak, for example, in a case where a body movement of the living body or a movement of something existing in the light path changes the illuminance on the measured plane in a measurement period.

The filter correction unit 162 in the present embodiment performs filter correction, in which a filter having a predetermined cycle property is adopted, both on the signal G that has been extracted by the signal extractor 150 and on the corrected signal C1. The filter correction unit 162 then outputs a corrected signal C2 and a corrected signal C1′. Examples of the corrected signal C2 will be described later.

In the present embodiment, the filter correction unit 162 performs the filter correction on the signal G, so that a noise component on a cycle different from the pulse wave cycle can be reduced.

In addition, in the present embodiment, the filter correction is performed on the corrected signal C1 in which the differential correction has been performed. Therefore, it is possible to reduce a noise component that has a cycle different from the pulse wave cycle and that has been increased by the differential correction.

The filter having a predetermined cycle property in the present embodiment may be a bandpass filter that passes only a frequency component approximate to the pulse wave cycle, for example. In the filter correction unit 162, the filter may employ a method of generating a filter coefficient having a cycle similar to the pulse wave cycle, calculating a mutual correlation with a target signal, and reducing a noise component having a cycle different from the pulse wave cycle.

As described above, the correction unit 160 in the present embodiment obtains the corrected signal C2 by performing the filter correction on the signal G, and the corrected signal C1′ by performing the filter correction on the corrected signal C1 in which the differential correction using the signal G and the signal R has been performed. Then, the correction unit 160 in the present embodiment outputs the corrected signal C2 and the corrected signal C1′ to the peak detection processing unit 170.

The peak detection processing unit 170 in the present embodiment includes a signal divider 171, an influence degree calculator 172, a corrected signal selector 173, and a peak time detector 174.

The signal divider 171 divides the corrected signal C2 and the corrected signal C1′ that have been output from the correction unit 160 into a plurality of segments, in units of the pulse wave cycle.

The influence degree calculator 172 calculates an influence degree of noise in each of the time segments. The influence degree herein serves as a value indicating a degree in the noise influence in each of the corrected signal C2 and the corrected signal C1′. The noise in the present embodiment includes an in-phase noise in the pulse wave cycle and an out-of-phase noise in the pulse wave cycle.

To be specific, in the present embodiment, the influence degree serves as a distortion amount indicating a waveform distortion in each of the corrected signal C2 and the corrected signal C1′. Hence, the influence degree calculator 172 in the present embodiment calculates the distortion amounts of the corrected signal C2 and the corrected signal C1′. The influence degree calculator 172 will be described later in detail.

The corrected signal selector 173 selects a corrected signal having a smaller one of the influence degrees that have been calculated by the influence degree calculator 172.

The peak time detector 174 detects a time when the peak occurs in the corrected signal that has been selected by the corrected signal selector 173. The peak time detector 174 may calculate the time when the peak occurs, a period between the peaks, and a change of the period between the peaks.

As described above, in the present embodiment, the corrected signal having a smaller noise influence is selected from the corrected signals obtained by performing a plurality of types of correction on the signal indicating the pulse wave, and the occurrence time of a pulse wave peak is detected from the corrected signal that has been selected.

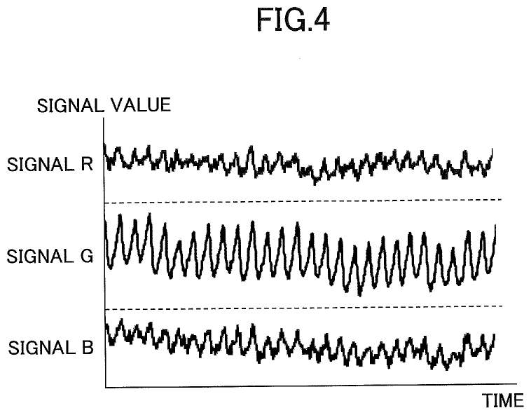

Next, referring to FIG. 4 to FIG. 6B, in the present embodiment, examples of the signal that has been extracted by the signal extractor 150 and examples of the signal that has been corrected by the correction unit 160 will be described.

FIG. 4 is an example of the RGB signals. In FIG. 4, an example of the RGB signals is illustrated when the signal extractor 150 extracts the signal R, the signal G, and the signal B from the image data.

In FIG. 4, as described above, cyclic changes in the signal values induced by changes of the pulse wave volume are found in each of the RGB signals. Among the signal R, the signal G, and the signal B, the signal G has the greatest change amount, whereas the signal R and the signal B have smaller change amounts.

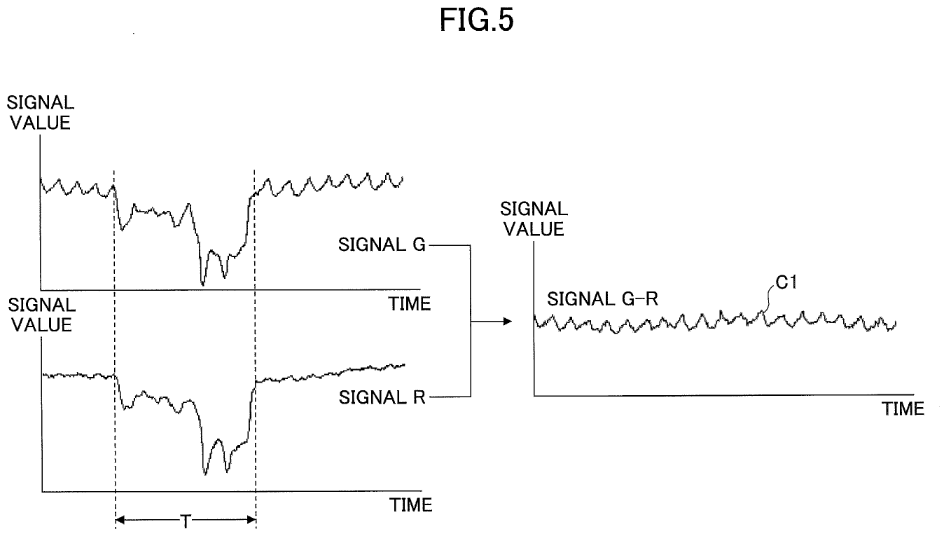

FIG. 5 illustrates an example of a differential correction result.

In FIG. 5, the corrected signal C1 is illustrated. The corrected signal C1 is a result of the differential correction that has been performed by the differential correction unit 161, by using the signal G and the signal R, in a time segment T.

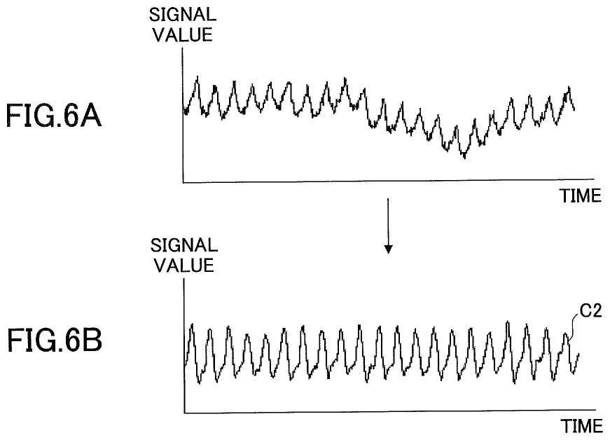

FIG. 6A and FIG. 6B illustrate an example of a filter correction result. FIG. 6A illustrates the signal G and FIG. 6B illustrates the corrected signal C2. The corrected signal C2 is the filter correction result in which the filter correction has been performed on the signal G by the filter correction unit 162.

In the example of FIG. 6A and FIG. 6B, in the pulse wave cycle of the signal G, the signal values change in longer cycles and in shorter cycles. However, it is understood that the above-described changes in the signal values are reduced in the corrected signal C2 in which the filter correction has been performed by the filter correction unit 162.

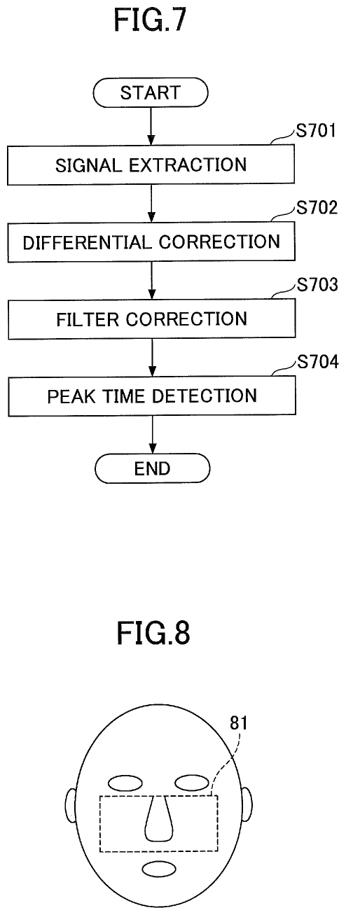

Next, referring to FIG. 7, an operation of the pulse wave measuring device 100 in the present embodiment will be described. FIG. 7 is a flowchart of the operation of the pulse wave measuring device.

In the pulse wave measuring device 100 in the present embodiment, the signal extractor 150 of the pulse wave measurement processing unit 130 extracts the signal indicating the pulse wave from the image data stored in the data storage 120 (step S701). To be specific, the signal extractor 150 acquires the image data of a predetermined measurement period stored in the data storage 120, extracts the signal G and the signal R, and outputs the signal G and the signal R to the correction unit 160.

Then, the differential correction unit 161 of the correction unit 160 performs the differential correction using the signal G and the signal R, and outputs the corrected signal C1 (step S702).

Subsequently, the filter correction unit 162 of the correction unit 160 performs the filter correction on the signal G that has been output from the signal extractor 150, and outputs the corrected signal C2. The filter correction unit 162 of the correction unit 160 also performs the filter correction on the corrected signal C1 that has been output from the differential correction unit 161, and outputs the corrected signal C1′ (step S703).

Next, the peak detection processing unit 170 of the pulse wave measurement processing unit 130 divides the corrected signal C2 and the corrected signal C1′ into segments, selects in each of the segments either one of the corrected signal C2 or the corrected signal C1′, and detects the peak of the corrected signal that has been selected and a time when the peak occurs (step S704).

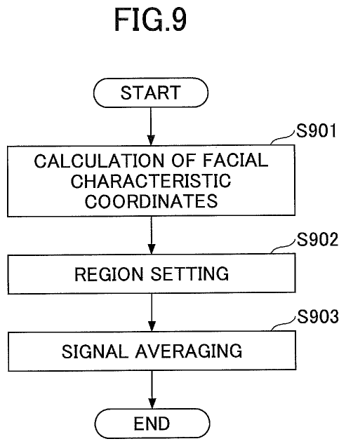

In the following, referring to FIG. 8 and FIG. 9, the process of the signal extractor 150 will be described further.

FIG. 8 illustrates acquisition of an image by the imaging unit. The signal extractor 150 in the present embodiment extracts the signal G and the signal R from the image data including a facial part of the living body 1, for example.

In the present embodiment, the imaging device 11 captures an image of the facial part of the measurement target (i.e., living body 1), from which the pulse waves are taken. From the captured image, the signal G and the signal R are extracted from image data of a region 81 including the nose and cheeks.

In the region 81, blood vessels pass near the skin surface, and a change in light amount reflected by the pulse wave can be easily observed in the captured image. When the pulse wave signal is extracted from the image including the region 81, an evaluation value in an analysis can be calculated with accuracy.

Additionally, in the region 81, the skin is less likely covered with hair or cloth. For this reason, when the pulse wave signal is extracted from the image including the region 81, the evaluation value in the analysis can be calculated with accuracy.

As described above, the RGB signal values in the region 81 cyclically change depending on a change in the hemoglobin amount in blood induced by the pulse wave. The pulse wave measurement processing unit 130 utilizes such a property and detects the peak in the signal indicating the pulse wave.

It is to be noted that the region 81 may not necessarily be contained in the region illustrated in FIG. 8. The region 81 may be any other region where changes in the RGB signals induced by the pulse wave can be observed. For example, a forehead part or a fingertip part can be a target region to capture the images. The region 81 may be any region to capture the images continuously in the measurement period.

Positions of characteristic parts such as eyes and nose may be detected in an image in accordance with a facial recognition technology, so that the pixel size of the region 81 may be set based on these positions in the image. Additionally, the input device 14 of the pulse wave measuring device 100 may be used for inputting a start point in the pixel position and pixel numbers of the width and height of the region 81.

The measurement may include capturing of images, and the terminal device or the imaging device included in the terminal device may measure and capture the images from a position apart from the living body in a so-called non-contact manner.

FIG. 9 is a flowchart of a process of a signal extractor.

The signal extractor 150 in the present embodiment calculates coordinates of the facial characteristic point in the image data that has been acquired from the data storage 120 (step S901). To be specific, the signal extractor 150 detects the facial parts, and respectively detects the coordinate positions of the characteristic points including eyes, nose, and mouth, for example, from a facial image. In the detection of the facial parts, any existing facial recognition technology may be used.

Then, the signal extractor 150 sets the region 81 to be used for detecting the pulse wave based on the coordinate positions of the characteristic points that have been detected in step S901 (step S902). The width of the region 81 can be set within a range that is not outside the facial region with coordinate positions of the nose being the center. The height of the region 81 can be set within a range that does not overlap eyes or nose.

Subsequently, the signal extractor 150 adds and averages the values of the signal G in the region 81 (step S903). The change in the reflected light amount in the region 81 induced by the pulse wave is a minute one. In a single pixel, there is a great influence caused by noise. However, by adding and averaging the signal values of a plurality of pixels, the noise influence on the signal indicating the pulse wave is reduced.

In the present embodiment, the above process is applied on all of the image data (i.e., all frames) in the image data group that has been acquired from the data storage 120, and the signal G and the signal R are extracted from the data in each image.

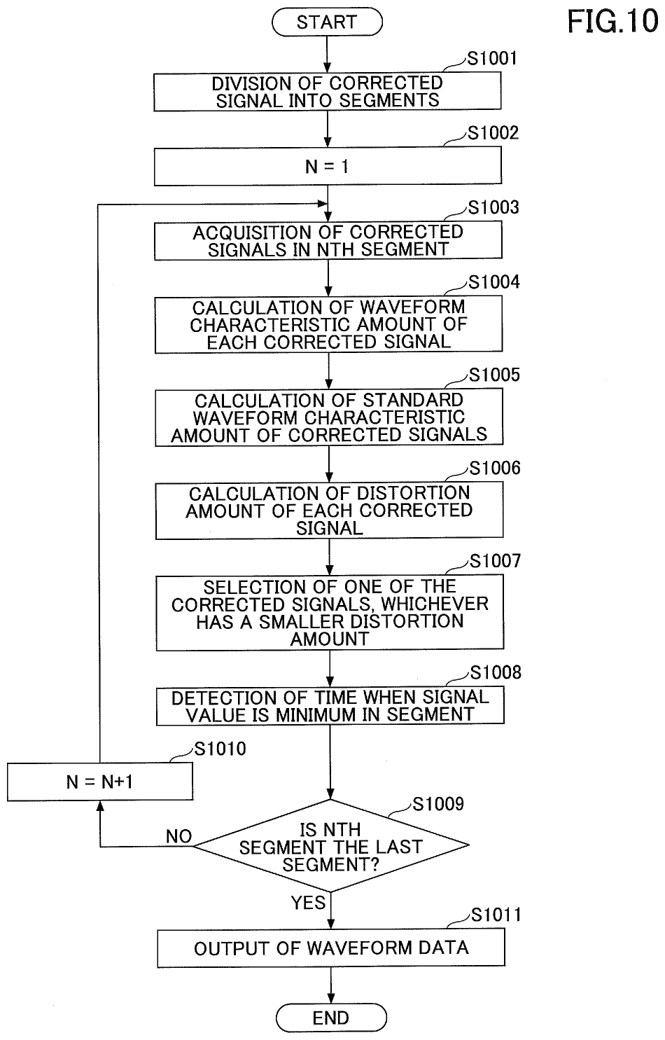

Next, referring to FIG. 10, the process of the peak detection processing unit 170 in the present embodiment will be described. FIG. 10 is a flowchart of the process of the peak detection processing unit in the first embodiment.

The signal divider 171 of the peak detection processing unit 170 in the present embodiment divides the corrected signal C2 and the corrected signal C1′ that have been output from the correction unit 160 into segments of the pulse wave cycles (step S1001).

As for the pulse wave cycle, both of the corrected signal C2 and the corrected signal C1′ take the local maximum values in a leading period of the pulse wave when the hemoglobin amount in the blood is the minimum. This property is utilized for detecting the pulse wave cycle. That is to say, the signal divider 171 in the present embodiment divides the corrected signal C2 and the corrected signal C1′ into segments, so that each of the divided segments corresponds to one cycle of the pulse wave indicated by (Tmax(i), Tmax(i+1)). Tmax(i) represents the (i)th local maximum time and Tmax(i+1) represents the (i+1)th local maximum time in the corrected signal C2 and the corrected signal C1′.

Then, the peak detection processing unit 170 sets 1 to a variable N, which is N=1 (step S1002). The peak detection processing unit 170 acquires the corrected signal C2 and the corrected signal C1′ of an Nth segment (step S1003).

Subsequently, the influence degree calculator 172 of the peak detection processing unit 170 performs step S1004 to step S1006, and calculates the influence degree of the noise on the corrected signal C2 and the corrected signal C1′. The influence degree in the present embodiment may include distortion amounts of the corrected signal C2 and the corrected signal C1′.

The influence degree calculator 172 of the peak detection processing unit 170 calculates waveform characteristic amounts of the corrected signal C2 and the corrected signal C1′ (step S1004). Then, the influence degree calculator 172 calculates standard waveform characteristic amounts of the corrected signal C2 and the corrected signal C1′ (step S1005).

Subsequently, the influence degree calculator 172 calculates the distortion amounts of the corrected signal C2 and the corrected signal C1′ using the waveform characteristic amounts and the standard waveform characteristic amounts (step S1006). The detailed processes in calculating the waveform characteristic amount, the standard waveform characteristic amount, and the distortion amount will be described later.

Then, the corrected signal selector 173 of the peak detection processing unit 170 selects a smaller one of the distortion amounts of the corrected signals (step S1007).

Next, the peak detection processing unit 170 detects the time when the corrected signal that has been selected has the minimum value in the Nth segment, and sets the detected time as the time when the peak occurs (step S1008).

Subsequently, the peak detection processing unit 170 determines whether the Nth segment is the last segment (step S1009). In step S1009, when the Nth segment is not the last segment, the peak detection processing unit 170 increments the variable N=N+1 (step S1010), and then returns to step S1003.

In step S1009, when the Nth segment is the last segment, the peak detection processing unit 170 outputs information indicating the occurrence time of the pulse wave peak for each of the segments in the measurement period (step S1011). Then, the peak detection processing unit 170 ends the process.

In the data storage 120, pulse wave data may be stored together with the information indicating the occurrence time of the peak in each of the segments. Herein, the pulse wave data includes waveform data in which the corrected signals that have been selected for the segments are connected. Upon receipt of an acquisition request from, for example, an external device arranged in the exterior of the pulse wave measuring device 100, the data stored in the data storage 120 can be output from the data output unit 140.

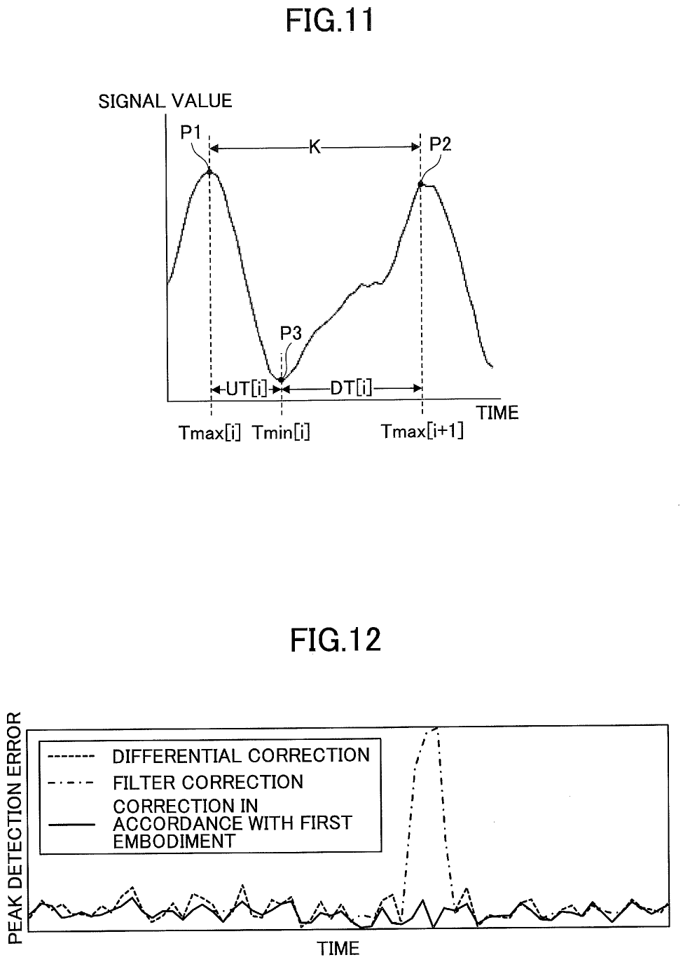

Hereinafter, referring to FIG. 11, the calculation of the influence degree will be described in detail. FIG. 11 is a diagram illustrating the process of calculating the influence degree.

The waveform illustrated in FIG. 11 indicates the corrected signal C2 in a segment K, for example. In the present embodiment, the cycle of the pulse wave is set to one segment, which corresponds to a period from a local maximum value P1 to a next local maximum value P2 of the corrected signal C2.

In the present embodiment, in the segment K, a point P3 is the minimum value of the corrected signal C2 and is detected as a peak.

Firstly, the calculation of the waveform characteristic amount of the corrected signal C2 in the segment K will be described. In the present embodiment, a ratio DT(i)/UT(i) of a leading period UT(i) and a trailing period DT(i) of the corrected signal C2 is used as the waveform characteristic amount. The leading period UT(i) and the trailing period DT(i) are calculated in the following expressions (2).

UT(i)=Tmin(i)−Tmax(i)

DT(i)=Tmax(i+1)−Tmin(i) (2)

Tmin(i) represents the time when the corrected signal C2 has the minimum value in the time segment (Tmax(i), Tmax(i+1)) (i.e., segment K).

Next, the calculation of the standard waveform characteristic amount in the present embodiment will be described. In the present embodiment, the waveform characteristic amount UT(i)/DT(i) in each segment is averaged for all segments and the averaged value is set to the standard waveform characteristic amount. The standard waveform characteristic amount UT/DT is calculated by the following expression (3).

UT/DT=Σ(UT(i)/DT(i))/N (3)

Next, the calculation of the distortion amount in the present embodiment will be described. In the present embodiment, the difference between the waveform characteristic amount and the standard waveform characteristic amount is set to the distortion amount. The distortion amount Δ(UT/DT)(i) is calculated in the following expression (4).

Δ(UT/DT) (i)=|UT/DT−UT(i)/DT(i)| (4)

In the present embodiment, as described above, the waveform characteristic amounts and the standard waveform characteristic amounts of the corrected signal C2 and the corrected signal C1′ in each segment are calculated, and the distortion amounts are calculated.

In the present embodiment, in each segment, one of the corrected signal C2 and the corrected signal C1′, whichever has a smaller distortion amount is selected for the corrected signal having a smaller noise influence degree.

When there is a small in-phase noise influence in the pulse wave cycle, the corrected signal C2, in which the filter correction has been performed and there is no drop in the waveform sensitivity and no increase in the out-of-phase noise, tends to be smaller in the distortion amount of the waveform than the corrected signal C1′ on which the differential correction has been performed. On the other hand, when there is a large in-phase noise influence in the pulse wave cycle, the corrected signal C1′, in which the differential correction that can reduce the in-phase noise in the pulse wave cycle has been performed, tends to be smaller in the distortion amount of the waveform than the corrected signal C2 in which the filter correction has been performed.

That is to say, the distortion amount of the corrected signal C2 is smaller than the distortion amount of the corrected signal C1′, when there is a small in-phase noise influence in the pulse wave cycle. The distortion amount of the corrected signal C1′ is smaller than the distortion amount of the corrected signal C2, when there is a large in-phase noise influence in the pulse wave cycle.

Hence, in the present embodiment, one of the corrected signal C2 and the corrected signal C1′, whichever has a smaller distortion amount is selected as a signal used for detecting the peak. Thus, the corrected signal having a smaller noise influence is selected.

As described above, in the present embodiment, the noise influence degrees of both of the corrected signal C2 and the corrected signal C1′ are calculated for each of the pulse wave cycles. In the present embodiment, the corrected signal having a smaller noise influence is selected for each pulse wave cycle, and the selected one is used for detecting the peak.

Which one of the corrected signal C2 or the corrected signal C1′ has a smaller noise influence changes depending on the magnitude of the in-phase noise influence in the pulse wave cycle. Therefore, in the present embodiment, the accuracy of detecting the peak can be improved in the entire measurement period, even if the magnitude of the in-phase noise influence in the pulse wave cycle changes depending on a body movement of the living body 1 whose pulse waves are being measured, the presence or absence of a movement of something existing in the light path, or the degree of the movement.

In other words, in the present embodiment, the influence caused by a disturbance can be suppressed, even when the pulse wave measuring device 100 acquires the signal indicating the pulse wave is in a non-contact state where the pulse wave measuring device 100 is not in contact with the living body 1. This configuration improves the accuracy of detecting the peak in the signal indicating the pulse wave.

In the present embodiment, in a predetermined measurement period, the image data that have been captured by the imaging device 11 is stored in the data storage 120. The signal extractor 150 is configured to extract the signal G and the signal R from the image data in which the measurement has been finished, but the present embodiment is not limited to this configuration.

The pulse wave measurement processing unit 130 in the present embodiment may perform the process of detecting the peak in parallel with the image capturing of the imaging device 11.

In this case, for the standard waveform characteristic amount, the influence degree calculator 172 may set additional average values of the waveform characteristic amounts of the corrected signal C2 and the corrected signal C1′ that have been acquired until the time when the signal extractor 150 extracts the signal G and the signal R. Also in this case, the standard waveform characteristic amount may be calculated in an experiment that has been conducted before, for example, and may be stored in the data storage 120 beforehand.

FIG. 12 is a diagram illustrating advantages of the first embodiment. In FIG. 12, errors of comparison results are illustrated. The comparisons are made between the occurrence times of the peaks that have been detected in the cases where the differential correction has been performed, the filter correction has been performed, and the present embodiment has been applied, and the occurrence times of the peaks that have been detected by a waveform measuring device measuring in a contact manner.

In the following description, the occurrence time of the peak detected by such a waveform measuring device of contact type is referred to as true occurrence time. In the example of FIG. 12, the peak detection errors are plotted on the vertical axis to represent standardized values of the errors between the true occurrence times of the peaks and the occurrence times of the peaks that have been detected in the cases where the differential correction has been performed, the filter correction has been performed, and the present embodiment has been applied.

In FIG. 12, in many time segments, the number of peak detection errors in the case where the filter correction has been performed is smaller than the number of peak detection errors in the case where the differential correction has been performed. This is because in the case where the filter correction has been performed, there is no drop in the sensitivity of the pulse wave and no increase in the out-of-phase noise that can be a problem in the differential correction, and the peak detection error is small in many time segments where the influence of the in-phase noise is small.

In the filter correction, however, in the time segment where the in-phase noise has great influence, the peak detection error is large and the accuracy of detecting the peak remarkably degrades. This also causes a large error in the change of the period between peaks.

In contrast, in the case where the present embodiment has been applied, in a time segment where the accuracy of detecting the peak degrades due to the filter correction, the peak is detected from the corrected signal in which the differential correction has been performed. This configuration of the present embodiment reduces or minimizes the degradation in the peak detection error.

### Second Embodiment

Next, a second embodiment will be described with reference to the drawings. The second embodiment is different from the first embodiment in that the value indicating the influence degree of in-phase noise contained in the noise is used for the influence degree. In the following description of the second embodiment, only the differences from the first embodiment will be described. Like numerals of the first embodiment are assigned to similar functional components of the second embodiment, and the description will be omitted.

The corrected signal C2 in which the filter correction has been performed includes in-phase noise in the pulse wave cycle and out-of-phase noise in the pulse wave cycle. In the corrected signal C2 in which the filter correction has been performed, noise (e.g., out-of-phase noise) having a cycle other than the pulse wave cycle has been reduced.

In the corrected signal C1′ in which the differential correction has been performed, the in-phase noise has been removed and only the out-of-phase noise in the pulse wave cycle remains. The out-of-phase noise in the corrected signal C1′ is greater than the out-of-phase noise in the corrected signal C2.

When there is a great influence of the in-phase noise in the pulse wave cycle, it can be said that the corrected signal C2 in which the noise includes the in-phase noise has a greater noise influence than the corrected signal C1′ has.

When there is a small influence of the in-phase noise in the pulse wave cycle, it can be said that the corrected signal C1′ in which the noise does not include the in-phase noise and the out-of-phase noise has not been reduced has a greater noise influence than the corrected signal C2 has.

Therefore, in the present embodiment, the corrected signal having a smaller noise influence is selected based on the influence degree of in-phase noise, and an occurrence time of a peak is detected from the corrected signal that has been selected. In other words, in the present embodiment, the value indicating the influence degree of in-phase noise is calculated as the influence degree, and the corrected signal having a smaller noise influence is selected based on the influence degree.

The influence degree calculator 172 in the present embodiment detects the maximum value and the minimum value of the signal R in each time segment, and calculates a change amount ΔR serving as a value indicating the influence degree of the in-phase noise.

The value of the change amount ΔR of the signal R tends to be larger, as the influence degree of in-phase noise becomes greater. The present embodiment gives attention on this tendency. While the change amount ΔR of the signal R is smaller than a predetermined threshold value, the influence of the in-phase noise is determined to be small and the corrected signal C2 in which the filter correction has been performed is used for detecting the peak. Also in the present embodiment, while the change amount ΔR of the signal R is equal to or larger than the predetermined threshold value, the influence of the in-phase noise is determined to be great and the corrected signal C1′ in which the differential correction has been performed is used for detecting the peak.

In the present embodiment, the corrected signal is selected as described above, and the corrected signal having a smaller noise influence is used for detecting the peak.

It is to be noted that in the present embodiment, a change amount value having the smallest peak detection error may be obtained in an experiment that has been conducted before, for example, and the obtained value may be set as the threshold value beforehand.

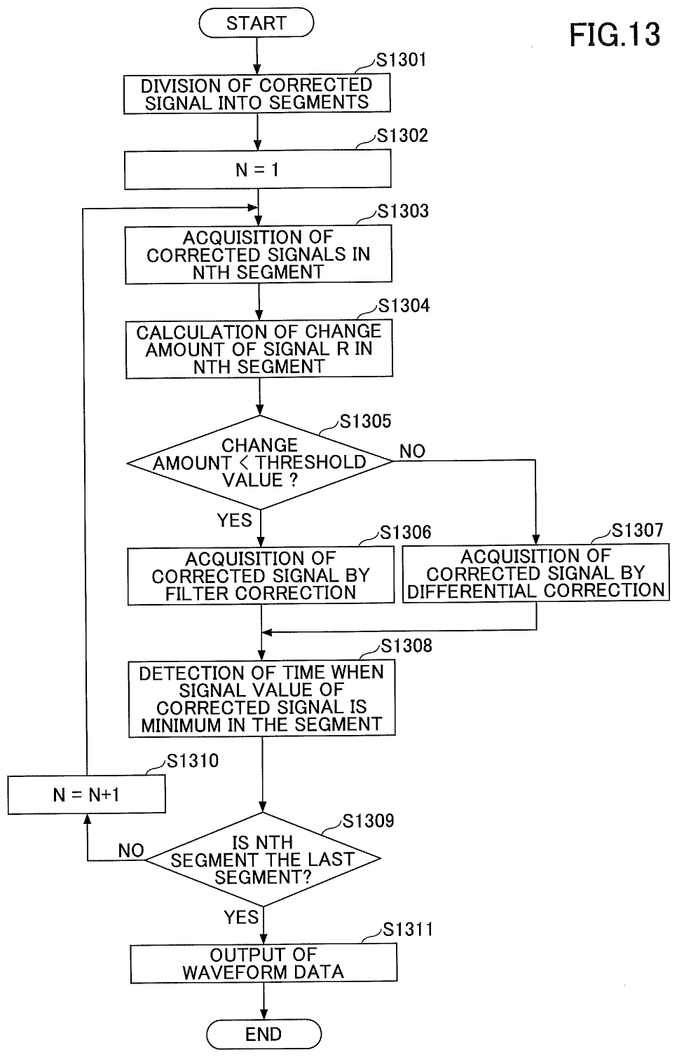

FIG. 13 is a flowchart of the process of the peak detection processing unit in the second embodiment.

The process in step S1301 to step S1303 in FIG. 13 is same as the process in step S1001 to step S1003 in FIG. 10, and the description will be omitted.

In step S1303, the corrected signal C1′ and the corrected signal C2 in the Nth time segment are acquired. Then, the influence degree calculator 172 calculates the change amount ΔR of the signal R in the Nth time segment (step S1304). In detail, the influence degree calculator 172 acquires the maximum value and the minimum value of the signal R in the Nth time segment that has been extracted by the signal extractor 150, and sets the difference between the maximum value and the minimum value to the change amount ΔR.

Subsequently, the influence degree calculator 172 determines whether the change amount ΔR is smaller than the threshold value (step S1305).

In step S1305, when the change amount ΔR is smaller than the threshold value, the corrected signal selector 173 selects the corrected signal C2 in which the filter correction has been performed (step S1306), and then the process goes to step S1308 to be described later. In step S1305, when the change amount ΔR is equal to or larger than the threshold value, the corrected signal selector 173 selects the corrected signal C1′ in which the differential correction has been performed (step S1307), and then the process goes to step S1308 to be described later.

Process from step S1308 to step S1311 is same as the process from step S1008 to step S1011 of FIG. 10, and the description will be omitted.

As described above, in the present embodiment, the value indicating the influence degree of in-phase noise is set to the change amount ΔR of the signal R for each time segment. This configuration achieves advantages similar to the advantages of the first embodiment.

It is to be noted that instead of the signal R, the signal B or the signal G may be used for calculating the change amount for each time segment. However, the signal having a smaller change in the sensitivity in the pulse wave can be used for determining the influence degree of in-phase noise.

### Third Embodiment

Next, a third embodiment will be described with reference to the drawings. The third embodiment is different from the second embodiment in that the value indicating the noise influence degree is calculated as a change amount in a distance signal indicating a distance between the living body 1 and the imaging device 11. In the following description in the third embodiment, only the differences from the second embodiment will be described. Like numerals of the second embodiment are assigned to similar functional components of the third embodiment, and the description will be omitted.

In the present embodiment, the imaging device 11 is configured to have a function of detecting the distance between the living body 1 and the imaging device 11.

In the present embodiment, the imaging device 11 may be a three-dimensional (3D) camera, for example. The imaging device 11 may include an infrared projector that irradiates an infrared light pattern for distance measurement and an infrared image sensor that receives an infrared reflection light from a target, so as to be capable of detecting the distance. In this case, the infrared light pattern that has been irradiated onto the living body 1 is analyzed, and a two-dimensional (2D) distance image can be acquired together with an RGB image.

When a 3D camera is used for the imaging device 11, any 3D camera of pattern projection (e.g., Structured Light) may be used, or any 3D camera such as a Time of Flight (TOF) camera or a stereo camera may be used for acquiring the distance image.

In the present embodiment, the region in the living body 1 captured by the imaging device 11 and the method of deciding the region are same as those in the first embodiment. The frame rate of the distance image in the present embodiment is 30 fps, which is same with the frame rate of the RGB image. The number of bits in a signal used for indicating the distance is 16 bits in the present embodiment. The optimal values of camera parameters such as the frame rate and the number of bits may be set in consideration of measurement environments, camera performances, and subsequent data processing, in a similar manner to the first embodiment.

In the present embodiment, the imaging device 11 may not be necessarily a 3D camera. Any imaging device capable of detecting the signal indicating the distance between the living body 1 and the imaging device 11 is applicable. In the following description, the signal indicating the distance between the living body 1 and the imaging device 11 is referred to as a distance signal. The distance signal is acquired by the image acquiring unit 110, for example, and is stored in the data storage 120 together with the RGB image data.

The influence degree calculator 172 in the present embodiment detects the maximum value and the minimum value of the distance signal in each time segment, and calculates a change amount ΔD.

The influence degree of in-phase noise tends to be greater, as the change amount ΔD of the distance signal becomes larger. The present embodiment gives attention to this tendency. In a time segment while the change amount ΔD of the distance signal is smaller than a predetermined threshold value, the influence of the in-phase noise in the pulse wave cycle is determined to be small and the corrected signal C2 in which the filter correction has been performed is used for detecting the peak. Also in the present embodiment, in another time segment while the change amount ΔD of the distance signal is equal to or larger than the predetermined threshold value, the influence of the in-phase noise is determined to be great and the corrected signal C1′ in which the differential correction has been performed is used for detecting the peak.

In the present embodiment, the corrected signal is selected as described above, and the corrected signal having a smaller noise influence is used for detecting the peak.

It is to be noted that in the present embodiment, a value of the change amount having the smallest peak detection error may be obtained in an experiment that has been conducted before, for example, and the obtained value may be set as the threshold value beforehand.

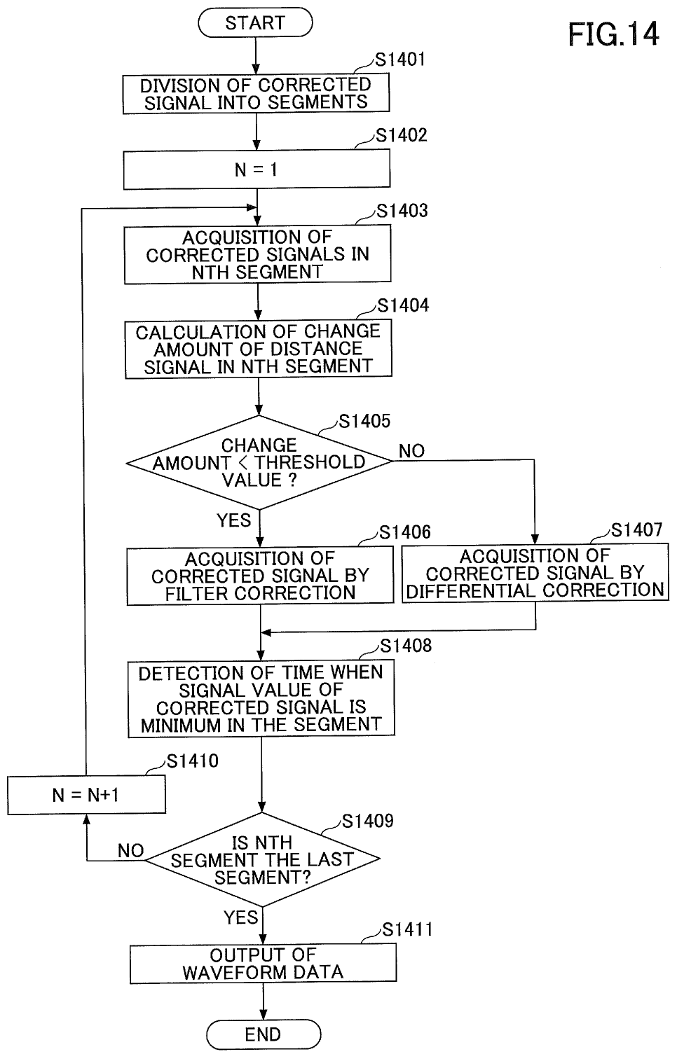

FIG. 14 is a flowchart of the process of the peak detection processing unit in the third embodiment.

Process in step S1401 to step S1403 in FIG. 14 is same as the process in step S1001 to step S1003 of FIG. 10, and the description will be omitted.

In step S1403, the influence degree calculator 172 acquires the corrected signal C1′ and the corrected signal C2 in the Nth time segment, and then calculates the change amount ΔD of the distance signal in the Nth time segment (step S1404). In detail, the influence degree calculator 172 acquires the distance signal in the Nth time segment, and sets the difference between the maximum value and the minimum value to the change amount ΔD.

Subsequently, the influence degree calculator 172 determines whether the change amount ΔD is smaller than the threshold value (step S1405). In step S1405, when the change amount ΔD is smaller than the threshold value, the corrected signal selector 173 selects the corrected signal C2 in which the filter correction has been performed in step S1406, and then the process goes to step S1408.

In step S1405, when the change amount ΔD is equal to or larger than the threshold value, the corrected signal selector 173 selects the corrected signal C1′ in which the differential correction has been performed in step S1407, and then the process goes to step S1408.

The process in step S1408 to step S1411 is the same as the process in step S1008 to step S1011 of FIG. 10, and the description is omitted.

As described above, in the present embodiment, the value indicating the influence degree of in-phase noise is set to the change amount ΔD of the distance signal for each time segment. This configuration achieves advantages similar to the advantages of the first embodiment.

### Fourth Embodiment

Hereinafter, a fourth embodiment will be described with reference to the drawings. The fourth embodiment is different from the first embodiment in that the corrected signal C1, which is output from the differential correction unit 161, is also supplied to the peak detection processing unit 170. In the following description in the fourth embodiment, only the differences from the first embodiment will be described. Like numerals of the first embodiment are assigned to similar functional components of the fourth embodiment, and the description is omitted.

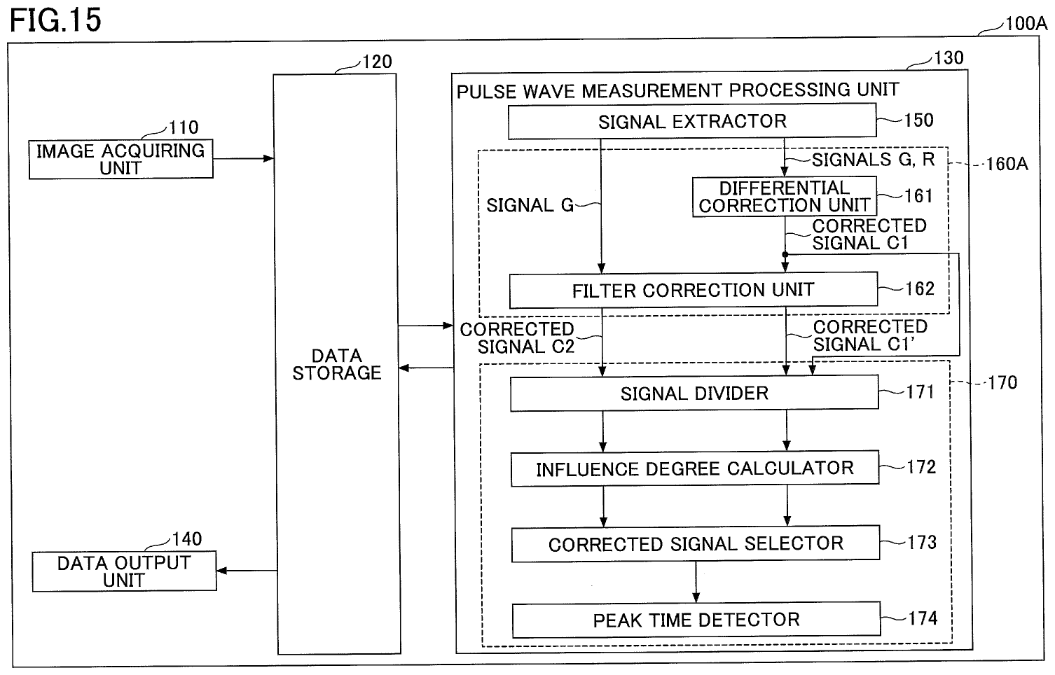

FIG. 15 is a diagram illustrating a functional configuration of the pulse wave measuring device in the fourth embodiment. In the present embodiment, a pulse wave measuring device 100A includes a correction unit 160A.

The correction unit 160A in the present embodiment supplies the corrected signal C1, which is output from the differential correction unit 161, to the signal divider 171 of the peak detection processing unit 170.

The peak detection processing unit 170 in the present embodiment performs the same process as in the first embodiment on the three corrected signals C1, C1′, and C2.

The peak detection processing unit 170 in the present embodiment respectively divides the three corrected signals C1, C1′, and C2 into time segments, and calculates the distortion amounts. The peak detection processing unit 170 detects the peak using one of the three corrected signals C1, C1′, and C2 having the smallest distortion amount.

In the present embodiment, the peak can be detected by using the corrected signal having the smallest noise influence. This configuration improves the accuracy of detecting the peak.

### Fifth Embodiment

Next, a fifth embodiment will be described with reference to the drawings. The fifth embodiment is different from the first embodiment in that the pulse wave measurement processing unit is arranged in the exterior of the terminal device including the imaging device. In the following description of the fifth embodiment, only the differences from the first embodiment will be described. Like numerals of the first embodiment are assigned to similar functional components of the fifth embodiment, and the description is omitted.

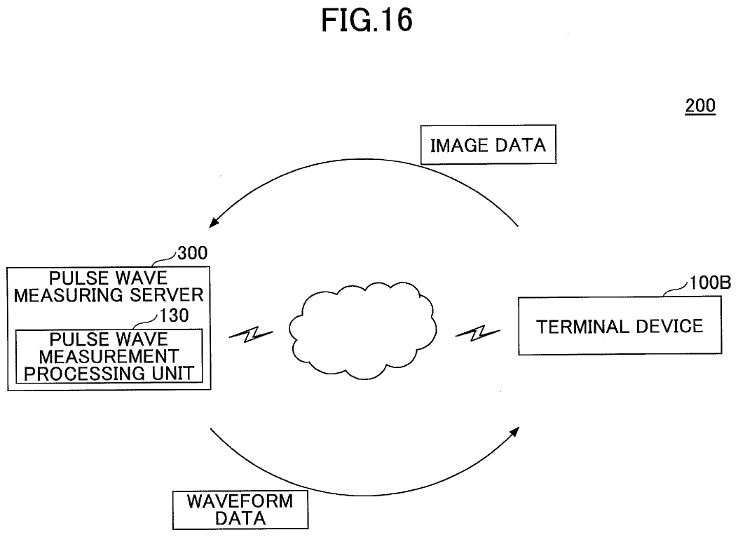

FIG. 16 illustrates a pulse wave measuring system in the fifth embodiment. A pulse wave measuring system 200 in the present embodiment includes a pulse wave measuring server 300 and a terminal device 100B.

The pulse wave measuring server 300 in the present embodiment is a generally-used information processing device including a CPU, a storage, and the pulse wave measurement processing unit 130. The terminal device 100B in the present embodiment includes the imaging device 11.

In the pulse wave measuring system 200 in the present embodiment, the terminal device 100B transmits the image data that has been captured by the imaging device 11 to the pulse wave measuring server 300.

In the pulse wave measuring server 300, the pulse wave measurement processing unit 130 performs the process using the image data that have been transmitted from the terminal device 100B, and acquires pulse wave data. The pulse wave measuring server 300 transmits the acquired pulse wave data to the terminal device 100B.

In the present embodiment, as described above, the pulse wave measurement processing unit 130 is included in the pulse wave measuring server 300, which is arranged in the exterior of the terminal device 100B. This configuration reduces burdens in processing of the terminal device 100B. Therefore, the terminal device 100B may have a simple configuration to be capable of transmitting the image data that have been captured by the imaging device 11. Examples of the terminal device 100B in the present embodiment may include, but are not limited to, an imaging device 11 having a transmission function.

In the example of FIG. 16, the pulse wave data that have been acquired by the pulse wave measuring server 300 are transmitted to the terminal device 100B, but the present embodiment is not limited to this configuration. The pulse wave measuring server 300 may transmit the pulse wave data to any device other than the terminal device 100B.

In the present embodiment, the example in which the pulse wave measuring server 300 includes the pulse wave measurement processing unit 130 has been described. However, the present embodiment is not limited to this configuration. The pulse wave measuring server 300 may include, for example, only the peak detection processing unit 170 of the pulse wave measurement processing unit 130, and the signal extractor 150 and the correction unit 160 may be included in the terminal device 100B.

### Sixth Embodiment

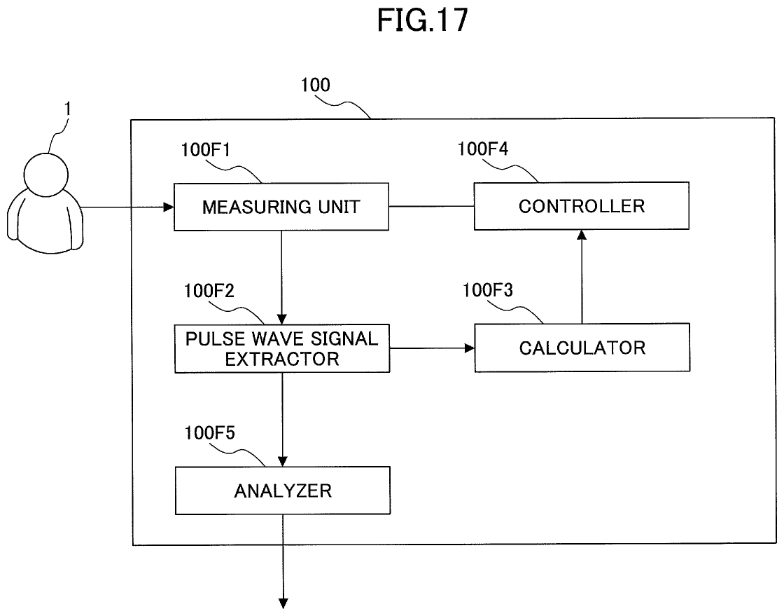

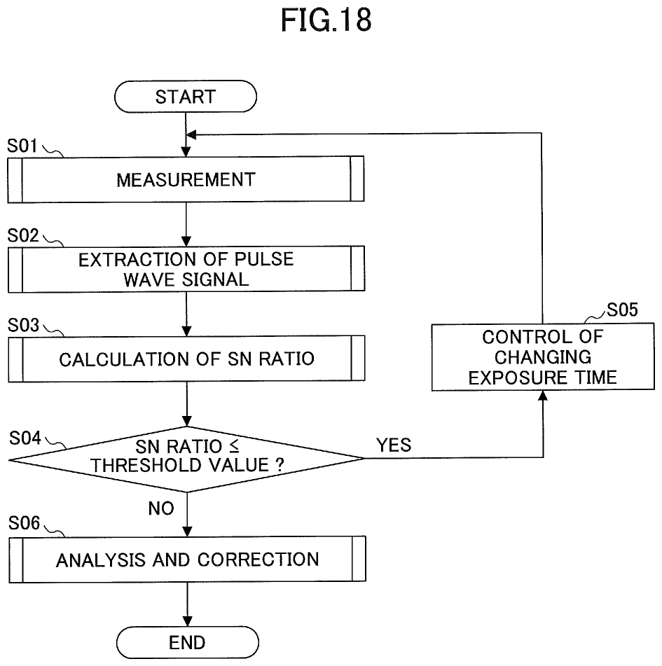

The pulse wave measuring device in a sixth embodiment measures the pulse wave, for example, as illustrated in FIG. 17, in a similar manner to the first embodiment. In addition, the pulse wave measuring device in the sixth embodiment is achieved by, for example, hardware similar to the hardware of the first embodiment. In the following description, only the differences will be described and the overlapping description will be omitted.

In the present embodiment, as illustrated in FIG. 17, the imaging device 11 (e.g., camera) embedded in the terminal device 100 is an example of the pulse wave measuring device. The imaging device 11 captures images of the living body 1, which is the measurement target from which the pulse waves are taken. From the captured image, the signals indicating the pulse waves of the living body 1 are acquired. In this case, the terminal device 100 in the present embodiment calculates a Signal to Noise (SN) ratio of the signal. When the SN ratio is equal to or smaller than a threshold value, the terminal device 100 controls an exposure time. In this manner, the terminal device 100 in the present embodiment is configured to capture an image having a high SN ratio. Then, the terminal device 100 in the present embodiment performs the peak detection process to detect an occurrence time of each peak in the pulse wave signal indicating the pulse wave.

In the present embodiment, the accuracy of detecting a pulse wave peak is improved, accordingly.

Also in the present embodiment, the improved accuracy of detecting the pulse wave peak improves the accuracy of detecting a change of the period between the peaks.

In the present embodiment, change information indicating the change of the period between the peaks can be given to a device that performs the process using the change information. The present embodiment allows highly accurate information indicating the change of a period between peaks to be available to a device that performs a process using such information. This configuration contributes to an improvement in the reliability of the process results of the process using the change information. Examples of the process using the change information may include, but are not limited to, a process of analyzing the operation of the autonomic nerve in a living body.

Functional Configuration Example

FIG. 17 is a block diagram illustrating an example of of a functional configuration of the pulse wave measuring device in the sixth embodiment. As illustrated in FIG. 17, the terminal device 100 includes a measuring unit 100F1, a pulse wave signal extractor 100F2, a calculator 100F3, a controller 100F4, and an analyzer 100F5.

The measuring unit 100F1 samples the light reflected from the living body. The living body is the target from which the pulse waves are measured. For example, the measuring unit 100F1 captures and generates images of the living body 1. The measuring unit 100F1 is achieved by the imaging device 11 (see FIG. 2), for example.

The pulse wave signal extractor 100F2 receives an image input from the measuring unit 100F1, and extracts a pulse wave signal from the image input from the measuring unit 100F1. For example, the pulse wave signal extractor 100F2 reads out every frame of each image that has been sampled by the measuring unit 100F1, and extracts a pulse wave signal from a signal value indicating the signal G mainly. The pulse wave signal extractor 100F2 is achieved by the CPU 12 (see FIG. 2), for example.

The calculator 100F3 calculates the SN ratio. In detail, the calculator 100F3 evaluates a signal component and a noise component of the pulse wave signal that has been extracted by the pulse wave signal extractor 100F2, and calculates an SN ratio. The calculator 100F3 is achieved by the CPU 12 (see FIG. 2), for example.

The control unit 100F4 controls each piece of hardware. For example, when the measuring unit 100F1 is achieved by a camera, the controller 100F4 sets either one or both of the frame rate and shutter speed of the camera to change the imaging conditions of the camera. In addition, the controller 100F4 controls each piece of hardware based on the SN ratio that has been calculated by the calculator 100F3. The controller 100F4 is achieved by the CPU 12 (see FIG. 17), for example.

The analyzer 100F5 analyzes the pulse wave signal that has been extracted by the pulse wave signal extractor 100F2, and evaluates a fluctuation in, for example, the pulse rate or the period between the peaks. The analyzer 100F5 is achieved by the CPU 12 (see FIG. 2), for example.

The terminal 100 may further include a correction unit.

Overall Process Example

FIG. 18 is a flowchart of an example of the overall process of the pulse wave measuring device in the sixth embodiment.

Measurement Example (step S01)

In step S01, the terminal device 100 measures the living body. For example, in step S01, the terminal device 100 captures an image of the living body, and generates an image. The image capturing is the same as in the first embodiment. That is, the terminal device 100 captures an image of the region 81 illustrated in FIG. 8, and generates an image.

Extraction Example of Pulse Wave Signal (step S02)

In step S02, the terminal device 100 extracts the pulse wave signal. For example, the extraction is same as in step S701 illustrated in FIG. 7. That is, in step S02, for example, the process as illustrated in FIG. 9 is performed.

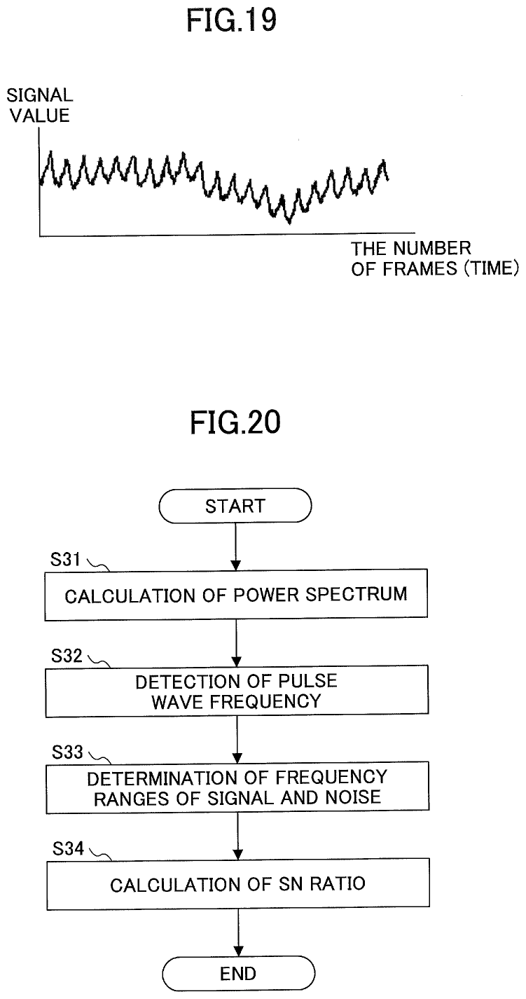

FIG. 19 is a diagram illustrating an example of the pulse wave signal in the sixth embodiment. In the example illustrated in FIG. 19, 30 pulse wave signals (per minute) are extracted. In FIG. 19, the horizontal axis represents the number of frames. Specifically, when a constant number of frames are captured in a predetermined period, the horizontal axis corresponds to time. On the other hand, the vertical axis represents signal value, which is an average value calculated in the process illustrated in FIG. 9. As illustrated in FIG. 19, a cyclically changing state depending on the pulse can be observed in the pulse wave signal.

Calculation Example of SN Ratio (step S03)

Referring back to FIG. 18, in step S03, the terminal device 100 calculates the SN ratio.

FIG. 20 is a flowchart of an example of the process of calculating the SN ratio in the sixth embodiment. For example, in step S03 illustrated in FIG. 18, the process illustrated in FIG. 20 will be performed.

Calculation Example of Power Spectrum (step S31)

In step S31, the terminal device 100 calculates a power spectrum. In detail, in step S31, the terminal device 100 firstly converts the number of data to be a power of 2. Next, the terminal device 100 performs Fast Fourier Transform (FFT) on the converted data. Then, the terminal device 100 calculates the power in each frequency.

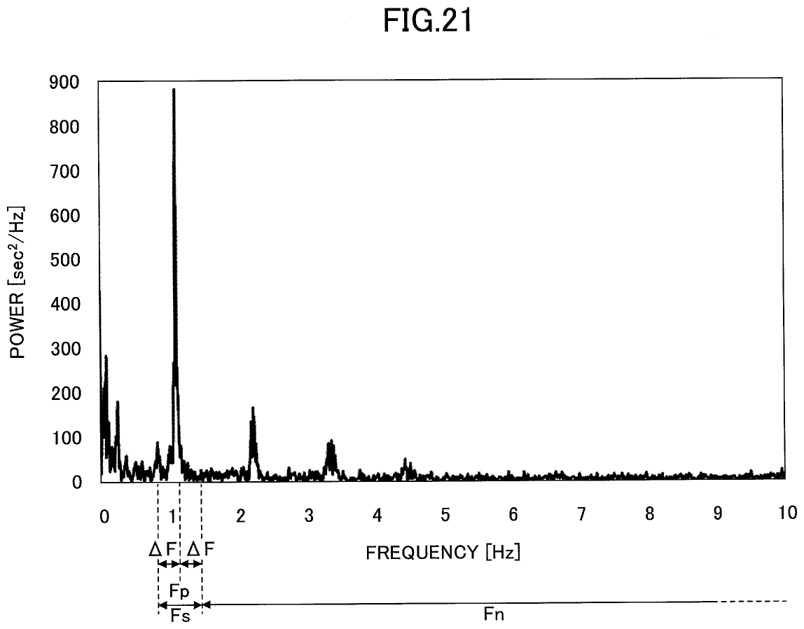

FIG. 21 is a graph of an example of the power spectrum calculated through calculation of the SN ratio in the sixth embodiment. For example, the graph of FIG. 21 is generated in step S31. In FIG. 21, the vertical axis represents power, whereas the horizontal axis represents frequency.

Detection Example of Pulse Wave Frequency (step S32)

Referring back to FIG. 20, in step S32, the terminal device 100 detects the pulse wave frequency. Next, an example where the power spectrum of FIG. 21 is calculated in step S31 will be described. In this example, a pulse wave frequency Fp is detected as illustrated in FIG. 21.

When a living body is in a resting state, the pulse rate ranges from 30 to 120 per minute in most cases. Hence, in the power spectrum, a fundamental wave having a strong peak is detected in accordance with the cycle of the number of pulse waves. That is, in a frequency range of from 0.5 Hz to 2.0 Hz, the terminal device 100 detects a frequency having the greatest power in the power spectrum. In this manner, the terminal device 100 is capable of detecting the pulse wave frequency Fp as illustrated in FIG. 21. More specifically, in the example of FIG. 21, the strong peak is detected in the frequency of about 1.0 Hz. In this example, the frequency of about 1.0 Hz is detected as the pulse wave frequency Fp, accordingly.

Determination Example of Frequency Ranges of Both Signal and Noise (step S33)

Referring back to FIG. 20, in step S33, the terminal device 100 determines frequency ranges of both the signal and the noise. For example, the frequency range of a signal component is determined by centering on the pulse wave frequency detected in step S32. To be specific, FIG. 21 illustrates an example of determining a frequency range Fs of the signal component to be a frequency range between “Fp−ΔF” and “Fp+ΔF”. In FIG. 21, “ΔF” is an example of 0.2 Hz. “ΔF” is a predetermined value. For example, “ΔF” can be set in consideration of the peak shape of the power spectrum. The frequency range Fs of the signal component may include a frequency range having a harmonic peak appearing in a frequency of an integral multiple of the pulse wave frequency Fp.

In step S33, on the other hand, the terminal device 100 determines a frequency range Fn of a noise component to be a frequency range other than the frequency range Fs of the signal component. It is to be noted that the power in a frequency lower than the pulse wave frequency Fp is influenced by, for example, a body movement of the living body while the living body is being measured. Therefore, the terminal device 100 may determine a frequency range higher than the frequency range Fs of the signal component to be the frequency range Fn of the noise component. The frequency range Fn of the noise component may include a frequency range lower than the frequency range Fs of the signal component. The upper limit frequency of the frequency range Fn of the noise component is “sampling frequency/2”. In detail, when the frame rate of the camera is “30 fps (the number of frames per second)”, for example, the upper limit frequency of the frequency range Fn of the noise component is “30 Hz/2=15 Hz”.

Calculation Example of SN Ratio (step S34)

Referring back to FIG. 20, in step S34, the terminal device 100 calculates the SN ratio. Specifically, in step S34, the terminal device 100 firstly calculates both the signal component “Vs” and the noise component “Vn” based on the frequency range of the signal component and the frequency range of the noise component that have been determined in step S33. The signal component “Vs” and the noise component “Vn” are respectively calculated by averaging the powers in the frequency ranges. Then, the terminal device 100 calculates the SN ratio, which is a ratio of “Vs” to “Vn” that have been calculated. The SN ratio may be calculated in any other calculation method.

Determination Example of Whether SN Ratio is Equal to or Smaller than Threshold Value (step S04)

Referring back to FIG. 18, in step S04, the terminal device 100 determines whether the SN ratio is equal to or smaller than a threshold value. In detail, the terminal device 100 compares the SN ratio that has been calculated in step S03 with the threshold value, and determines whether the SN ratio is equal to or smaller than the threshold value.

When the terminal device 100 determines that the SN ratio is equal to or smaller than the threshold value (Yes in step S04), the process goes to step S05. On the other hand, when the terminal device 100 determines that the SN ratio is larger than the threshold value (No in step S04), the process goes to step S06. When the terminal device 100 determines that the SN ratio is equal to or smaller than the threshold value, the terminal device 100 may display an alarm, for example, to stop the process of the pulse wave measurement. Then, the process may go to step S05.

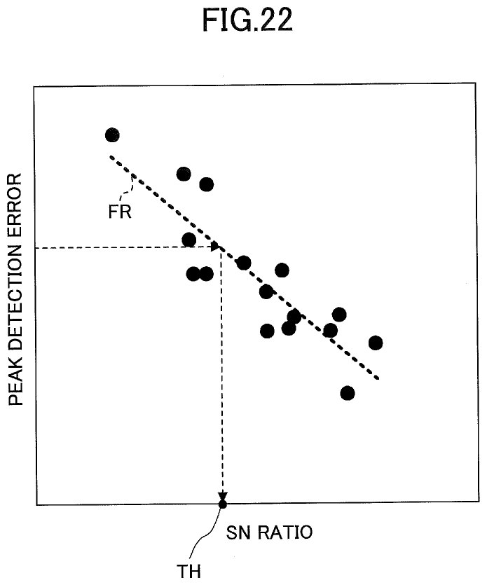

FIG. 22 is a graph of a setting example of the threshold value in the sixth embodiment. It is to be noted that FIG. 22 illustrates an example of a relationship between the SN ratio and the peak detection error.

In FIG. 22, the horizontal axis represents value of the SN ratio calculated in the calculation method in step S34 of FIG. 20, for example. The vertical axis represents an error between the peak time detected in the analysis of the terminal device 100 and the true peak time that is a peak time detected by a waveform measuring device of contact type.

As illustrated in FIG. 22, a relationship can be seen such that as the SN ratio is smaller, the peak detection error tends to be larger. Hence, the terminal device 100 firstly calculates a relational formula FR indicating the relationship between the peak detection error and the SN ratio. The relational formula FR is calculated, for example, in a least-square method based on each point.

Next, the terminal device 100 determines allowable peak detection errors, which are the values in the vertical axis in accordance with purposes of analysis to be performed in a later step. Then, based on on the relational formula FR, the terminal device 100 determines a threshold value TH corresponding to the determined vertical value. In this manner, the terminal device 100 sets the threshold value TH. The threshold value TH determined in this manner may be set beforehand in the terminal device 100 to perform step S04 of FIG. 18.

Control Example of Changing Exposure Time (step S05)

Referring back to FIG. 18, in step S05, the terminal device 100 controls changing of an exposure time.

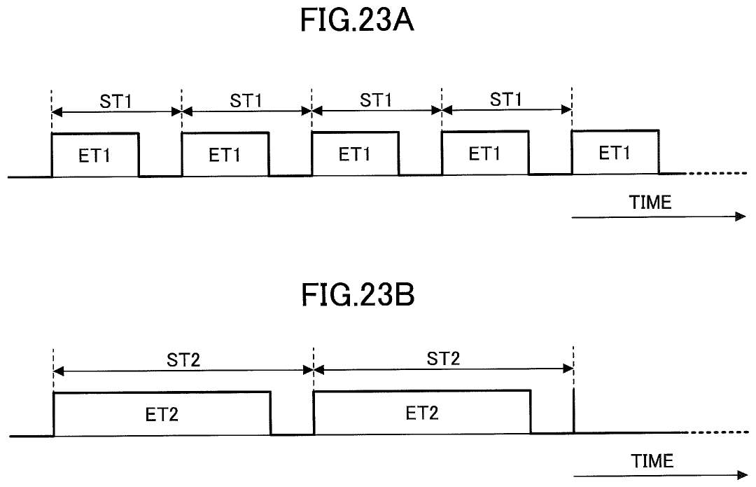

FIG. 23A and FIG. 23B are timing diagrams illustrating an example of control by the pulse wave measuring device in the sixth embodiment. In the following, the exposure time determined by initial settings of the shutter speed will be described as an example of the exposure time in FIG. 23A (hereinafter, referred to as “first exposure time ET1”). The cycle determined by the frame rate in the initial settings is assumed to be a sampling period (hereinafter, referred to as “first sampling cycle ST1”) illustrated in FIG. 23A. It is to be noted that the exposure time is a period of time while the camera shutter is being released and the light is being taken, that is a period of time while the exposure is being performed.

Next, when the terminal device 100 determines that the SN ratio is equal to or smaller than a predetermined threshold value (Yes in step S04 of FIG. 18), the terminal device 100 controls changing of the exposure time to another exposure time illustrated in FIG. 23B (hereinafter, referred to as “second exposure time ET2”), for example. In detail, the terminal device 100 changes a setting value of the shutter speed to control changing of the exposure time to the second exposure time ET2. As illustrated in FIG. 23B, the second exposure time ET2 is longer than the first exposure time ET1. Accordingly, as the exposure time is longer, the received light amount by each frame is increased. When the received light amount by each frame is insufficient, the signal component in the power spectrum is stronger. That is, as the exposure time is longer, the SN ratio can be increased. As to the relationship between the SN ratio and the peak detection error, the relationship illustrated in FIG. 22 can be often seen. Thus, the increased SN ratio reduces the peak detection error. Therefore, the terminal device 100 is capable of controlling changing of the exposure time to increase the SN ratio and reduce the peak detection error.

The terminal device 100 may slow the cycle to be set by the frame rate, which is an example of the cycle of sampling the pulse wave signal. In the example of FIG. 23A and FIG. 23B, the terminal device 100 controls changing of the first sampling cycle ST1 to a sampling cycle illustrated in FIG. 23B (hereinafter, referred to as “second sampling cycle ST2”). In one cycle, the exposure time cannot be set longer than the cycle to be set by the frame rate, in many cases. Therefore, the terminal device 100 controls changing of the frame rate, for example, so as to make the sampling cycle longer, like the second sampling cycle ST2 illustrated in FIG. 23B. Before such a change, in other words, in the case where the frame rate is 60 fps, for example, as illustrated in FIG. 23A, the terminal device 100 changes the frame rate to “30 fps”. Such a slowed frame rate allows the terminal device 100 to make the exposure time longer, like changing of the first exposure time ET1 to the second exposure time ET2 in one cycle.

Analysis and Correction Example (step S06)

Referring back to FIG. 18, in step S06, the terminal device device 100 performs analysis and correction.

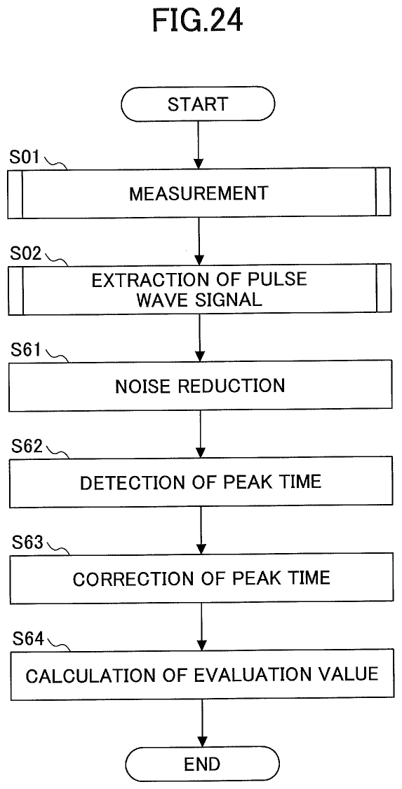

FIG. 24 is a flowchart of an example of analysis and correction by the wave pulse measuring device in one embodiment of the present invention. In FIG. 24, same processes as those in FIG. 18 have identical references and overlapping description will be omitted.

Measurement Example (step S01)

In step S01, in a similar manner to FIG. 18, the terminal device 100 captures images of a living body and measures the living body. In FIG. 24, the measurement may be performed for a predetermined period to meet purposes of analysis in a later step. For example, in order to calculate values of Low Frequency (LF) and High Frequency (HF) of the pulse wave signal, that is in order to calculate fluctuation values of both of LF and HF, the measurement may be conducted for about two minutes and data may be stored.

Detection Example of Pulse Wave Signal (step S02)

In step S02, for example, in a similar manner to FIG. 18, the terminal device 100 detects a pulse wave signal. Accordingly, in a similar manner to FIG. 18, the pulse wave signal illustrated in FIG. 19 is detected in step S02.

Noise Reduction Example (step S61)

In step S61, the terminal device 100 reduces noise in the pulse wave signal. A known technique may be used in a method for reducing the noise. One example of a method for reducing the noise may be a so-called filter correction technique in which a bandpass filter is applied. Another example of the method for reducing the noise may be a so-called differential correction technique in which the difference between two color signals is taken to reduce the in-phase noise generated by a body movement of a living body or a change in the illuminance while the measurement is being conducted.

The noise reduction results in an example illustrated in FIG. 6B.

Detection Example of Peak Time (step S62)

In step S62, the terminal device 100 detects the peak time. In detail, in step S62, the terminal device 100 detects the peak time from the pulse wave signal, which has been generated in step S61 and in which the noise has been reduced. Since hemoglobin absorbs light, the signal value becomes smaller at the peak. By utilizing this property, the peak time can be detected by calculating a time having the minimum value in a time segment, which is a period between the time when the signal value is the local maximum and the time when the signal value is the next local maximum, in each pulse wave.

Correction Example of Peak Time (step S63)

In step S63, the terminal device 100 corrects the peak time.

In step S05 illustrated in FIG. 18, to increase the SN ratio, the frame rate slows in some cases. In such cases, the slowed frame rate makes a temporal resolution insufficient depending on the purpose of analysis to be conducted in a later step. In other words, in such cases, the number of sampled signal values the pulse wave signal for a predetermined period is equal to or smaller than a predetermined value. For example, when the temporal resolution is insufficient, the terminal device 100 corrects the peak time.

In detail, the terminal device 100 approximates and corrects the peak time based on a plurality of signal values including a signal value of any peak and signal values of both before and after a peak. In the following, some correction examples of the peak time using three signal values will be described.

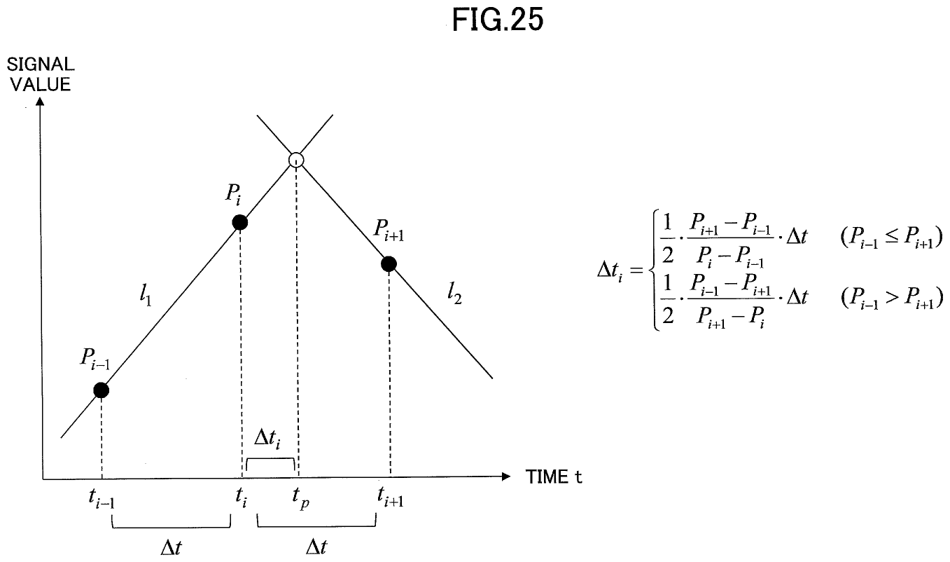

FIG. 25 is illustrates a correction example of the peak time by the pulse wave measuring device in one embodiment of the present invention. The illustrated correction is called broken line approximation. In this correction, a peak corrected amount Δti serves as the peak time obtained by the correction. A first signal value Pi serves as the peak time correction. A second signal value Pi−1 serves as the peak signal value immediately before the first signal value Pi. A third signal value Pi+1 serves as the peak signal value immediately after the first signal value Pi. In this case, the peak corrected amount Δti can be obtained by the expressions illustrated in FIG. 25, for example.

The peak corrected amount Δti is obtained by the expressions illustrated in FIG. 25, and then the terminal device 100 increases the temporal resolution of the pulse wave signal. In other words, the corrected peak time allows the terminal device 100 to increase the temporal resolution of the pulse wave signal. This configuration allows the terminal device 100 to calculate the peak time of the pulse wave with high accuracy, even from the pulse wave signal of a low temporal resolution.

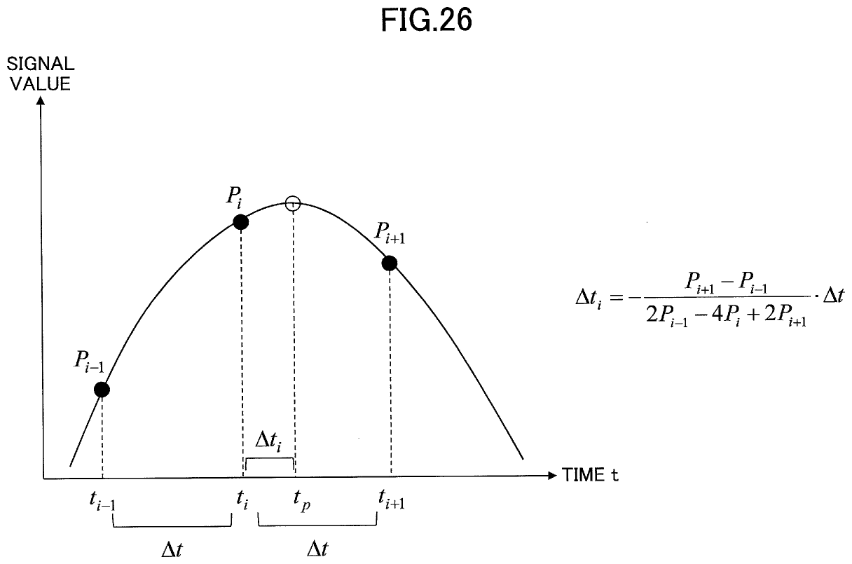

FIG. 26 is illustrates another correction example of the peak time by the pulse wave measuring device in one embodiment of the present invention. In step S63 illustrated in FIG. 24, the correction illustrated in FIG. 26 may be performed. Like the correction illustrated in FIG. 25, the peak corrected amount Δti serves as the peak time correction. Further, the first signal value Pi, the second signal value Pi−1, and the third signal value Pi+1 are same as illustrated in FIG. 25. In this correction, the peak corrected amount Δti is calculated in the expression illustrated in FIG. 26. The illustrated correction is called parabolic approximation.

The peak corrected amount Δti is obtained by the expression illustrated in FIG. 26, and then the terminal device 100 increases the temporal resolution of the pulse wave signal. In other words, as the peak time is corrected, the terminal device 100 is capable of increasing the temporal resolution of the pulse wave signal. Hence, the terminal device 100 calculates the peak time of the pulse wave with high accuracy, even from the pulse wave signal of a low temporal resolution.

The correction method is not limited to the methods illustrated in FIG. 25 and FIG. 26. For example, in the correction example of FIG. 25, it is assumed that inclinations of two straight lines are set to “l1” and “l2” that satisfies “l1=−l2”. In this regard, in most pulse waves, the inclination before the peak is steeper than the inclination after the peak. Therefore, by utilizing such a property, the terminal device 100 may correct the peak time to satisfy “l1>−l2”. This configuration allows the terminal device 100 to correct the peak time with higher accuracy.

In addition, any interpolation method such as spline interpolation may use used for the correction method. In addition, the number of signal values to be used for correction is not limited to three. Three or more signal values may be used by adding one or more different signal values near the three values.

The correction may be conducted, when the sampled number of signal values of the pulse wave signal for a predetermined period is equal to or smaller than a predetermined number. An example of the predetermine period is a unit of time, and may be “one second”. The predetermined value can be set based on the purpose of analysis in a later step. For a purpose of analyzing the operation of the autonomic nerve of a living body, for example, at least 256 signal values can be detected per second. In this case, the correction may be conducted so that the temporal resolution can be at least 256 per second.

Calculation Example of Evaluation Value (step S64)

Referring back to FIG. 24, in step S64, the terminal device 100 calculates an evaluation value for analysis. For example, the terminal device 100 calculates the evaluation value such as a pulse rate, a fluctuation between the pulse peaks, and a combination thereof, based on each peak time that has been detected.

In addition, in at least one embodiment of the present invention, one or more processes may be performed by any information processing device or any information processing system based on one or more programs. That is to say, at least one embodiment of the present invention may be implemented by one or more programs to cause a computer to carry out the pulse wave measuring method. It is to be noted that one or more programs are stored in a computer-readable recording medium to be installed in a computer.

Further, at least one embodiment of the present invention may be achieved by a pulse wave measuring system including a plurality of information processing devices including a terminal computer. That is, each of the embodiments may be achieved by the pulse wave measuring system illustrated in FIG. 6, for example.

It is to be noted that in the pulse wave measuring system, the terminal device 100B and the pulse wave measuring server 300 may perform the overall process by sharing the process in any other way than the ways that have been described heretofore. In the pulse wave measuring system, the process may be performed by a plurality of devices in a redundant, distributed, or parallel manner.

As described heretofore, the exposure time is controlled based on the SN ratio of the pulse wave signal to be extracted, and the pulse wave measuring device increases the SN ratio to reduce the peak detection error. By reducing the peak detection error in this manner, the pulse wave measuring device improves the accuracy of detecting the pulse wave peak.

Further, the present invention is not limited to these embodiments, but various variations and modifications may be made without departing from the scope of the present invention.

### RELATED ART DOCUMENT

### Patent Document

Patent Document 1: Japanese Translation of PCT International Application Publication No. JP-T-2014-529439
Patent Document 2: Japanese Patent No. 5332406

What is claimed is:

1. A pulse wave measuring device comprising:
a signal divider configured to divide a first corrected signal and a second corrected signal into a plurality of segments, respectively, each of which corresponds to a cycle of a pulse wave of a living body, the first corrected signal and the second corrected signal being results obtained through a first correction and a second correction respectively using a first signal and a second signal that are extracted from image data and that change in association with the pulse wave;
an influence degree calculator configured to calculate a value indicating a degree of a noise influence in each of the first corrected signal and the second corrected signal for each of the plurality of segments; and
a peak time detector configured to detect, separately for each of the plurality of segments, a time of occurrence of a peak in one of the first corrected signal and the second corrected signal that has a smaller degree of the noise influence for each of the plurality of segments.

2. The pulse wave measuring device according to claim 1, further comprising:
a first correction unit configured to reduce a noise component different from a component on a frequency of the pulse wave through the first correction, and to output the first corrected signal; and
a second correction unit configured to calculate a difference between the first signal and the second signal to reduce an in-phase noise between the first signal and the second signal through the second correction, and to output the second corrected signal.

3. The pulse wave measuring device according to claim 2, wherein the second corrected signal is output from the second correction unit, when the first signal is input into the second correction unit.

4. The pulse wave measuring device according to claim 1, wherein the value indicating the degree of the noise influence is a distortion amount of each of the first corrected signal and the second corrected signal in each of the plurality of segments.

5. The pulse wave measuring device according to claim 4, wherein the distortion amount is calculated in accordance with a ratio of a leading period to a trailing period, the leading period starting from a minimum value continuing to a maximum value, the trailing period starting from the maximum value continuing to a next minimum value, in each of the plurality of segments of each of the first corrected signal and the second corrected signal.

6. The pulse wave measuring device according to claim 2, further comprising:
a corrected signal selector configured to select the one of the first corrected signal and the second corrected signal that have been calculated by the influence degree calculator,
wherein the value indicating the degree of the noise influence indicates a degree of an in-phase noise influence, and includes a change amount in the second signal calculated from a maximum value and a minimum value of the second signal in each of the plurality of segments, and
wherein the corrected signal selector selects the first corrected signal when the change amount is smaller than a threshold value, and selects the second corrected signal when the change amount is equal to or larger than the threshold value.

7. The pulse wave measuring device according to claim 2, further comprising:
a corrected signal selector configured to select the one of the first corrected signal and the second corrected signal that have been calculated by the influence degree calculator,
wherein the value indicating the degree of the noise influence indicates a degree of an in-phase noise influence, and includes a change amount in a distance between the living body and an imaging device capturing the image data from the living body, and
wherein the corrected signal selector selects the first corrected signal when the change amount is smaller than a threshold value, and selects the second corrected signal when the change amount is equal to or larger than the threshold value.

8. The pulse wave measuring device according to claim 1, wherein the first signal includes a first channel having a first light sensitivity in a G wavelength, and the second signal includes a second channel having a second light sensitivity in an R wavelength.

9. A pulse wave measuring system comprising:
a signal divider configured to divide a first corrected signal and a second corrected signal into a plurality of segments, respectively, each of which corresponds to a cycle of a pulse wave of a living body, the first corrected signal and the second corrected signal being results obtained through a first correction and a second correction respectively using a first living body signal and a second living body signal that are acquired in a non-contact manner with a living body and that change in association with the pulse wave;
an influence degree calculator configured to calculate a value indicating a degree of a noise influence in each of the first corrected signal and the second corrected signal for each of the plurality of segments; and
a peak time detector configured to detect, separately for each of the plurality of segments, a time of occurrence of a peak in one of the first corrected signal and the second corrected signal that has a smaller degree of the noise influence.
